(19) United States
(12) Patent Application Publication
(10) Pub. No.: US 2016/0186265 A1
(43) Pub. Date: Jun. 30, 2016

(54) Methods for Typing HLA Alleles

(71) Applicant: CENTRE HOSPITALIER UNIVERSITARIE VAUDOIS, Laussane (CH)

(72) Inventors: Cyril Andre, Lausanne (CH); Giuseppe Pantaleo, Pully (CH)

(21) Appl. No.: 14/911,739

(22) PCT Filed: Aug. 14, 2014
(86) PCT No.: PCT/IB2014/063925

§ 371 Date: Feb. 12, 2016

Related U.S. Application Data
(60) Provisional application No. 61/866,788, filed on Aug. 16, 2013.
(60) Provisional application No. 61/866,057, filed on Aug. 15, 2013.

(51) Int. Cl. C12Q 1/68

(52) U.S. Cl. CPC C12Q 1/6883; C12Q 1/6881; C12Q 2600/156

(57) ABSTRACT

This disclosure relates to the typing of human leucocyte antigen (HLA) alleles. More particularly, the present invention relates to HLA typing as a method of identify patients at risk of a hypersensitivity reaction to drugs such as abacavir and/or to diagnose disease (e.g., Behçet's disease).

### RELATED APPLICATIONS

This application claims priority to U.S. Ser. No. 61/866,057 filed Aug. 15, 2013 and U.S. Ser. No. 61/866,788 filed Aug. 16, 2013, both of which are incorporated into this application in their entirety.

### FIELD OF THE DISCLOSURE

This disclosure relates to the typing of human leucocyte antigen (HLA) alleles. More particularly, the present invention relates to HLA typing as a method of identify patients at risk of a hypersensitivity reaction to drugs such as abacavir and/or to diagnose disease (e.g., Behçet's disease).

### BACKGROUND

The HLA complex of humans (major histocompatibility complex or MEC) is a cluster of linked genes located on chromosome 6. The HLA complex is classically divided into three regions: class I, II, and III regions Class I HLAs comprise the transmembrane protein (heavy chain) and a molecule of beta-2 microglobulin. The class I transmembrane proteins are encoded by the HLA-A, HLA-B and HLA-C loci. The function of class I HLA molecules is to present antigenic peptides (including viral protein antigens) to T cells. The MHC exhibits high polymorphism; more than 200 genotypical alleles of HLA-B have been reported. The alleles are typically identified by the HLA locus name, then the symbol “*”, and an even number of digits specifying the particular allele. The first two digits represent a group of alleles (e.g., HLA-B*51 or HLA-B*57) and the next two digits a synonymous allele (e.g., HLA-B*51:01 or HLA-B*57:15). Additional digits may be included to identify mutations. Commercially available MHC identification testing (e.g., “typing”) systems are typically “two digit” systems, meaning that the tests only identify alleles up to the first two digits (e.g., HLA-B*51 or HLA-B*57), or “four digit” systems (e.g., identifying, e.g., HLA-B*57:01).

The products encoded by the polymorphic HLA loci are commonly typed by serological methods for transplant and transfusion histocompatibility testing, and blood component therapy. Serological typing is based on reactions between characterized sera and the HLA gene products. Known techniques for histocompatibility testing include microlymphocytotoxicity and flow cytometry. Standard microlymphocytotoxicity for HLA antigen typing determines the HLA antigen profile of a subject's lymphocytes, using a panel of well characterized HLA antisera. An exemplary non-serological HLA typing system is the Luminex system which is a reverse polymerase chain reaction sequence specific oligonucleotide (PCR-SSO) that involves PCR amplification of targeted regions within the MHC class I or II regions with group specific primers, followed by a process of probing the amplicon with Luminex beads, each coated with sequence specific oligonucleotide probes to identify the presence or absence of specific alleles. The assignment of HLA type is then based on the reaction pattern observed, compared to patterns associated with published sequences. Such methods are rather complicated, time-consuming and very expensive. More recently, methods for analysis of HLA polymorphisms at the genetic level have been developed. Non-serological HLA typing methods include the use of DNA restriction fragment length polymorphism, or labelled oligonucleotides, to identify specific HLA DNA sequences.

Behçet's disease is a rare immune-mediated small-vessel systemic vasculitis that often presents with mucous membrane ulceration and ocular problems. As a systemic disease, it can also involve visceral organs such as the gastrointestinal tract, pulmonary, musculoskeletal, cardiovascular and neurological systems. This syndrome can be fatal due to ruptured vascular aneurysms or severe neurological complications. A large number of serological studies show a linkage between the disease and HLA-B*51. The presence of the HLA-B*51 in a patient is an indication and/or confirmation of Behçet's disease diagnosis. Improved systems for prospective HLA-B*51 screening are also desired by those in the field and would be of great use in clinical medicine.

Pharmacogenetic testing is not yet widely used in routine clinical practice to optimize drug choice or clinical management. Hypersensitivity reactions (HSR) are unexpected, immune (allergy)-like reactions that occur in a minority of patients treated with antiretroviral drugs. One such antiretroviral drug is abacavir is a nucleoside reverse-transcriptase inhibitor with activity against the human immunodeficiency virus (HIV), available for once-daily use in combination with other antiretroviral agents, that has shown efficacy, few drug interactions, and a favorable long-term toxicity profile. The most important observed adverse effect of abacavir that limits its use in therapy and mandates a high degree of clinical vigilance is an immunologically-mediated hypersensitivity reaction affecting 5 to 8% of patients during the first 6 weeks of treatment. Symptoms of a hypersensitivity reaction to abacavir include combinations of fever, rash, constitutional symptoms, gastrointestinal tract symptoms, and respiratory symptoms that become more severe with continued dosing. Immediate and permanent discontinuation of abacavir is mandated, resulting in a rapid reversal of symptoms. Subsequent re-challenge with abacavir is contraindicated, since it can result in a more severe, rapid, and potentially life-threatening reaction. An association between a diagnosis of hypersensitivity reaction to abacavir and carriage of the major histocompatibility complex class I allele HLA-B*57:01 was reported independently by two research groups and was subsequently corroborated by several independent studies. HLA-B*57:01 screening has been found to reduce the incidence of hypersensitivity reaction to abacavir. An exemplary testing system (four digit) for identifying individuals at risk is the COBAS® AmpliPrep/COBAS® TaqMan® HLA-B*5701 Screening Test (Roche). Improved systems for prospective HLA-B*57:01 screening to identify subjects at increase risk for drug hypersensitivity reaction are desired by those in the field and would be of great use in clinical medicine.

### BRIEF DESCRIPTION OF THE DRAWINGS

FIG. 1A-B. Selection of HLA B*51 primers and probe.

FIG. 2A-B. Selection of HLA B*57:01 primers and probe.

### SUMMARY

This disclosure relates to methods for identifying one or more loci having genetic polymorphisms. The methods provide the user the ability to specifically amplify the locus having the genetic polymorphisms using a conventional primer free of a fluorescent material and enables FHA allele typing in real time using a labelled probe which is able to specifically detect amplification of the locus. In some embodiments, improved systems for prospective HLA-B*57:01 screening to identify subjects at increase risk for drug hypersensitivity reaction. In some embodiments, systems for prospective HLA-B*51 (e.g., four digit) screening are provided. Other embodiments will be apparent to the skilled artisan from this disclosure.

### DETAILED DESCRIPTION

This disclosure relates to the typing of HLA alleles. The methods and reagents provided herein enable the detection of HLA alleles using a real-time polymerase chain reaction (PCR) for diagnostic and/or preventative purposes. The real-time PCR assays may be performed on nucleic acid (e.g., DNA, RNA) isolated from a patients sample using oligonucleotides that specifically bind and may be used to amplify certain HLA alleles and a labeled oligonucleotide for detecting the amplified nucleic acid molecules representing the HLA allele(s). HLA typing may be performed by analyzing the data obtained from the real-time PCR. In certain embodiments, this disclosure relates to methods for amplifying and detecting (e.g., typing) particular HLA-B haplotypes without typing other HLA-B alleles. In certain embodiments, typing of alleles may be achieved by: i) amplifying a relevant fragment of one or more alleles with at least one suitable set of oligonucleotides; ii) hybridizing the amplification product of step i) to at least one oligonucleotide that specifically hybridizes to a target region comprising one or more polymorphic nucleotides within said relevant fragment; and, iii) determining from the result of step ii) the absence or presence of the alleles in the sample. The disclosure provides oligonucleotides to be used in such methods of typing alleles. Compositions and kits comprising such oligonucleotides are also provided.

For instance, HLA-B*51 and/or subtypes thereof may be amplified and detected in order to, for example, diagnose patients having Behçet's disease. In some embodiments, the methods provide for the amplification and/or detection of 4-digit HLA-B*51 subtypes such as, for instance, (e.g., HLA-B*51:01, HLA-B*51:02, HLA-B*51:03, HLA-B*51:05, HLA-B*51:07, HLA-B*51:08, HLA-B*51:09, HLA-B*51:10, HLA-B*51:11N, HLA-B*51:12, HLA-B*51:13, HLA-B*51:14, HLA-B*51:15, HLA-B*51:21, HLA-B*51:22, HLA-B*51:26, HLA-B*51:27N, HLA-B*51:29, HLA-B*51:31, HLA-B*51:32, HLA-B*51:33, HLA-B*51:36, HLA-B*51:37, HLA-B*51:63 and/or HLA-B*51:65 (e.g., any of SEQ ID NOS. 1-25)) without also amplifying and/or detecting other alleles (e.g., other HLA-B alleles). In some embodiments, the amplifying may be performed using one or more oligonucleotides having binding specificity for HLA-B*51 (e.g., one or more of the HLA-B*51 subtypes). In preferred embodiments, an oligonucleotide primer and/or primer pair (e.g., at least one “forward” and at least one “reverse” primer) that amplifies any one or more of the 4-digit HLA-B*51 subtypes (e.g., HLA-B*51:01, HLA-B*51:02, HLA-B*51:03, HLA-B*51:05, HLA-B*51:07, HLA-B*51:08, HLA-B*51:09, HLA-B*51:10, HLA-B*51:11N, HLA-B*51:12, HLA-B*51:13, HLA-B*51:14, HLA-B*51:15, HLA-B*51:21, HLA-B*51:22, HLA-B*51:26, HLA-B*51:27N, HLA-B*51:29, HLA-B*51:31, HLA-B*51:32, HLA-B*51:33, HLA-B*51:36, HLA-B*51:37, HLA-B*51:63 and/or HLA-B*51:65 (e.g., any of SEQ ID NOS. 1-25)) without also amplifying and/or detecting other HLA-B alleles. In certain embodiments, the primer and/or primer pair may be used to amplify a nucleic acid molecule corresponding to at least a portion of exon 2 and/or exon 3 an HLA-B*51 allele (e.g., or HLA-B*51 subtype). It is preferred that such portion is found within each of the 4-digit HLA-B*51 subtypes of interest (e.g., HLA-B*51:01, HLA-B*51:02, HLA-B*51:03, HLA-B*51:05, HLA-B*51:07, HLA-B*51:08, HLA-B*51:09, HLA-B*51:10, HLA-B*51:11N, HLA-B*51:12, HLA-B*51:13, HLA-B*51:14, HLA-B*51:15, HLA-B*51:21, HLA-B*51:22, HLA-B*51:26, HLA-B*51:27N, HLA-B*51:29, HLA-B*51:31, HLA-B*51:32, HLA-B*51:33, HLA-B*51:36, HLA-B*51:37, HLA-B*51:63 and/or HLA-B*51:65 (e.g., any of SEQ ID NOS. 1-25)) such that a primer and/or primer pair may be used to amplify nucleic acid sequences corresponding to any of such subtypes. In some embodiments, such a primer and/or primer pair do not also amplify other HLA-B alleles (separately or together (e.g., in a multiplex reaction)). In others, additional and/or other particular subtypes may be amplified and/or detected to the exclusion of other alleles and/or subtypes. An exemplary primer pair that may be used to amplify such HLA-B*51 subtypes may be, for instance, SEQ ID NO.:40 (e.g., as a forward primer) and/or SEQ ID NO.:41 (e.g., as a reverse primer). The amplification of such HLA-B*51 subtypes may be detected by hybridizing the same to one or more nucleic acid probes corresponding to (e.g., binding to) to target regions within the nucleic acid molecules. Such target regions typically comprise one or more polymorphic nucleotide sequences within exon 2 and/or 3 of HLA-B*51. In some embodiments, the amplified nucleic acid may correspond to an HLA-B*51 subtype selected from the group consisting of HLA-B*51:01 (SEQ ID NO: 1), HLA-B*51:02 (SEQ ID NO: 2), HLA-B*51:03 (SEQ ID NO: 3), HLA-B*51:05 (SEQ ID NO: 4), HLA-B*51:07 (SEQ ID NO: 5), HLA-B*51:08 (SEQ ID NO: 6), HLA-B*51:09 (SEQ ID NO: 7), HLA-B*51:10 (SEQ ID NO: 8), HLA-B*51:11N (SEQ ID NO: 9), HLA-B*51:12 (SEQ ID NO: 10), HLA-B*51:13 (SEQ ID NO: 11) HLA-B*51:14 (SEQ ID NO: 12), HLA-B*51:15 (SEQ ID NO: 13), HLA-B*51:21 (SEQ ID NO: 14), HLA-B*51:22 (SEQ ID NO: 15), HLA-B*51:26 (SEQ ID NO: 16), HLA-B*51:27N (SEQ ID NO: 17), HLA-B*51:29 (SEQ ID NO: 18), HLA-B*51:31 (SEQ ID NO: 19), HLA-B*51:32 (SEQ ID NO: 20), HLA-B*51:33 (SEQ ID NO: 21), HLA-B*51:36 (SEQ ID NO: 22), HLA-B*51:37 (SEQ ID NO: 23), HLA-B*51:63 (SEQ ID NO: 24) and/or HLA-B*51:65 (SEQ ID NO: 25), and/or a fragment and/or derivative thereof. In some embodiments, the probe may be or may comprise SEQ ID NO.:42, for instance. Other embodiments of such assays for amplifying and/or detecting HLA-B*51 are also contemplated from this disclosure, as may be determined by one of ordinary skill in the art.

In some embodiments, the HLA allele amplified and/or detected may be HLA-B*57 (e.g., HLA-B*57:01 or another subtype thereof) to, for instance, identify an individual as being at risk of a hypersensitivity reaction to abacavir. In some embodiments, the methods provide for the amplification and/or detection of HLA-B*57:01 without also amplifying and/or detecting other alleles (e.g., other HLA-B*57 subtypes, separately or together (e.g., in a multiplex reaction)). In some embodiments, the amplifying may be performed using one or more oligonucleotides having binding specificity for HLA-B*57:01. In preferred embodiments, an oligonucleotide primer and/or primer pair (e.g., at least one “forward” and at least one “reverse” primer) that amplifies HLA-B*57:01 without also amplifying and/or detecting other HLA-B*57 subtypes. In certain embodiments, the primer and/or primer pair may be used to amplify a nucleic acid molecule corresponding to at least a portion of exon 3 of HLA-B*57:01. In some embodiments, such a primer and/or primer pair do not also amplify other HLA-B*57 subtypes. In others, additional and/or other particular subtypes may be amplified and/or detected to the exclusion of other alleles and/or subtypes. An exemplary primer pair that may be used to amplify such HLA-B*57 subtypes may be, for instance, SEQ ID NO.:43 (e.g., as a forward primer) and/or SEQ ID NO.:44 (e.g., as a reverse primer). The amplification of HLA-B*57 may be detected by hybridizing the same to one or more nucleic acid probes corresponding to (e.g., binding to) to target regions within the nucleic acid molecules. Such target regions typically comprise one or more polymorphic nucleotide sequences within exon 3 of HLA-B*57. In some embodiments, the amplified nucleic acid may correspond to HLA-B*57 subtype selected from the group consisting of HLA-B*57:01:01 (SEQ ID NO: 26), HLA-B*57:01:02 (SEQ ID NO: 27), HLA-B*57:01:03 (SEQ ID NO: 28), HLA-B*57:01:04 (SEQ ID NO: 29), HLA-B*57:01:05 (SEQ ID NO: 30), HLA-B*57:01:06 (SEQ ID NO: 31), HLA-B*57:01:07 (SEQ ID NO: 32), HLA-B*57:01:08 (SEQ ID NO: 33), HLA-B*57:01:09 (SEQ ID NO: 34), HLA-B*57:01:10 (SEQ ID NO: 35), and HLA-B*57:01:11 (SEQ ID NO: 36), and/or a fragment and/or derivative thereof. In some embodiments, the probe may be or may comprise SEQ ID NO.:45, for instance. Other embodiments of such assays for amplifying and/or detecting HLA-B*57:01 are also contemplated from this disclosure, as may be determined by one of ordinary skill in the art.

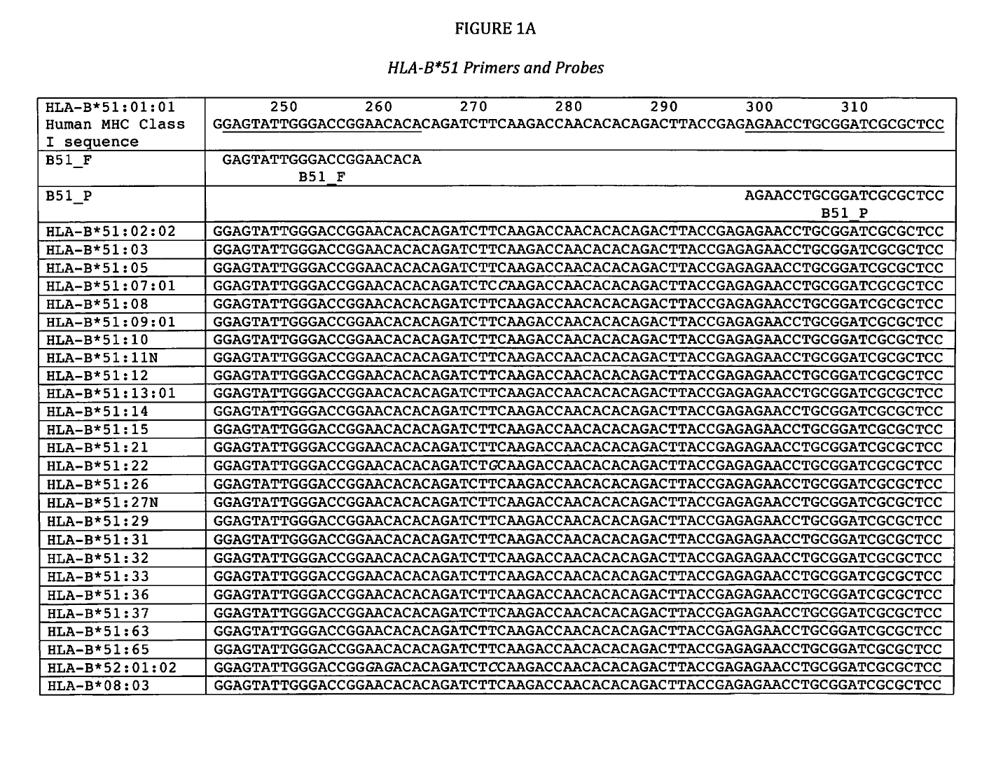

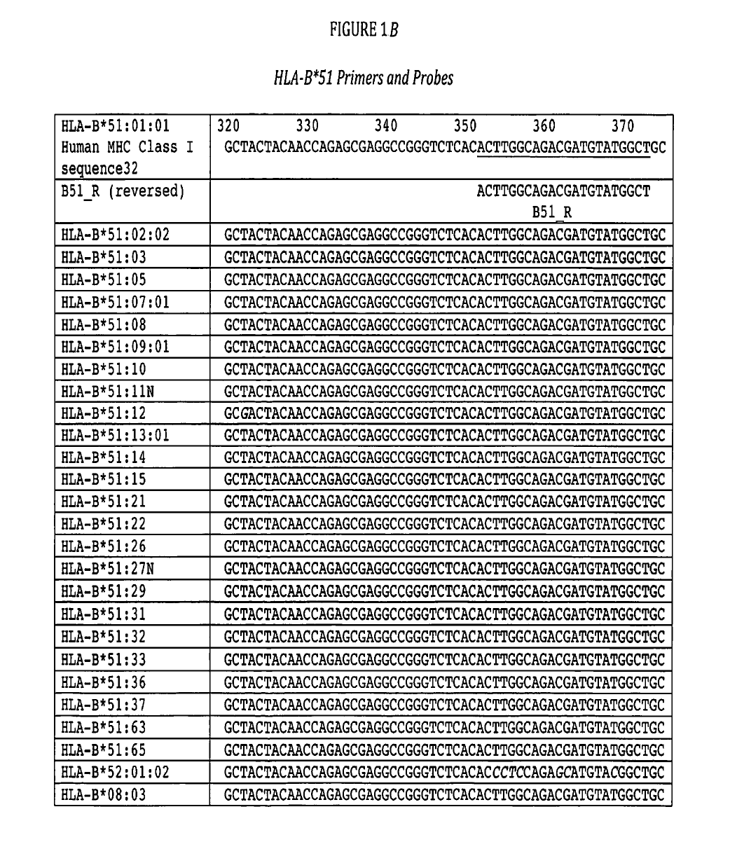

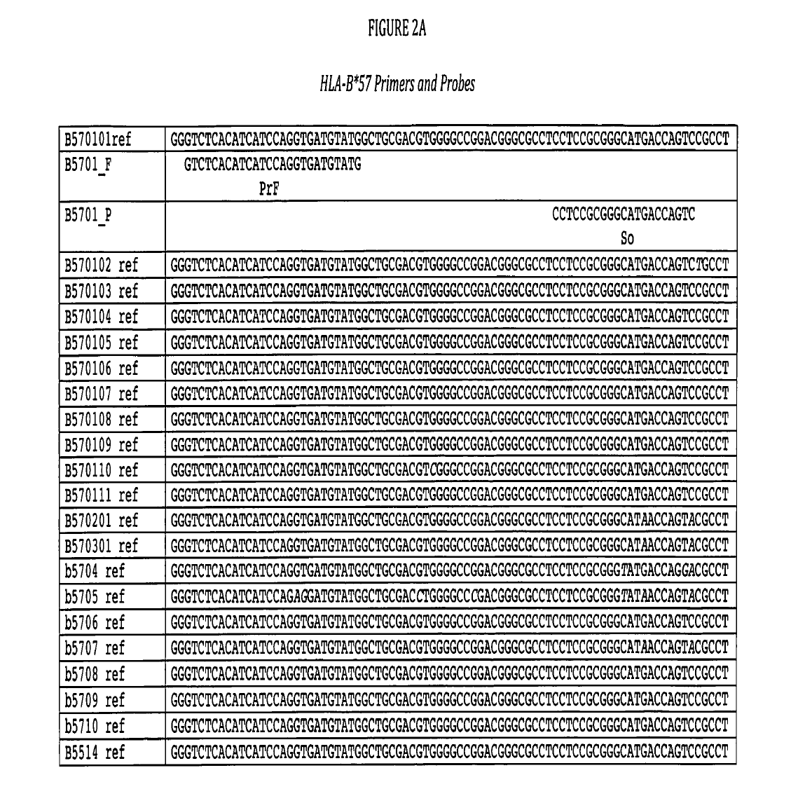

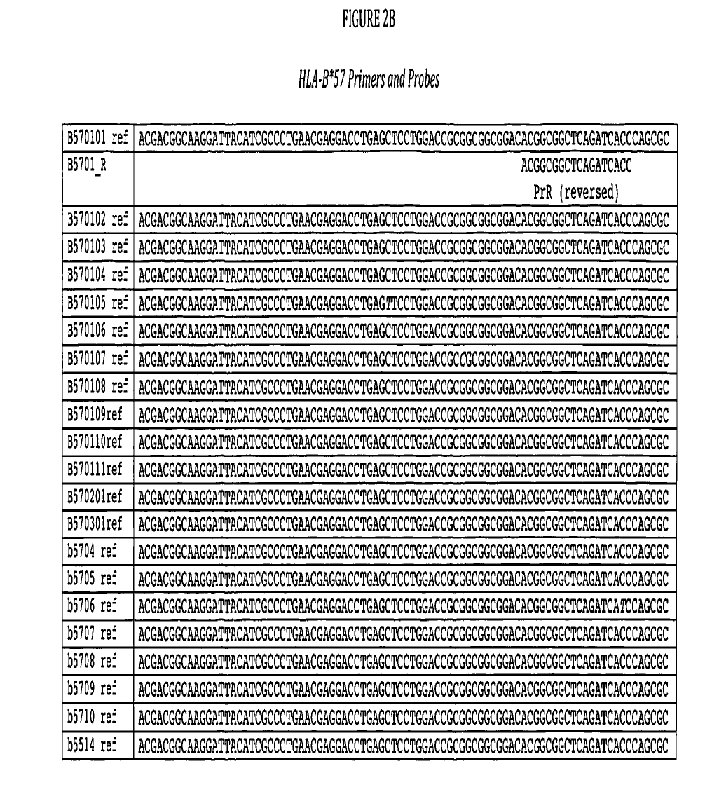

In certain embodiments, the one or more nucleic acid probes may be labeled with one or more detectable labels. The real-time PCR assays may be single and/or multiplex assays. The methods may also comprise the amplification of one or more control nucleic acid sequences. For example, control nucleic acid sequences may be used to amplify and detect a control human DNA target (ribonuclease 30 kDa (RPP30)) using, for example, SEQ ID NO.:37 (e.g., as a forward primer), SEQ ID NO.:38 (e.g., as a reverse primer) and/or SEQ ID NO.:39 (e.g., as an oligonucleotide probe). As described above, kits are also provided. Such kits may include, for instance, oligonucleotides having the sequence of one or more of SEQ ID NOS. 40, 41 and/or 42 for amplifying and/or detecting HLA-B*51 alleles (e.g., any of the above-described subtypes) and/or one or more of SEQ ID NOS. 43, 44 and/or 45 for amplifying and/or detecting HLA-B*57:01. The kits may also include one or more control samples and/or control oligonucleotides (e.g., one or more of SEQ ID NOS. 37, 38 and/or 39). The oligonucleotides (e.g., the oligonucleotide probes) may also comprise a detectable label. The kits may further comprise instructions for use in amplifying and/or detecting HLA-B*51 alleles (e.g., any of the above-described subtypes, FIG. 1) and/or HLA-B*57:01 (e.g., FIG. 2).

As used herein, the term “amplifying” typically refers to cycling polymerase-mediated exponential amplification of nucleic acids employing primers that hybridize to complementary strands, as described for example in Innis et al., PCR Protocols: A Guide to Methods and Applications, Academic Press (1990). The product of PCR amplification of DNA is typically referred to as an “amplicon”. Devices have been developed that can perform thermal cycling reactions with compositions containing fluorescent indicators which are able to emit a light beam of a specified wavelength, read the intensity of the fluorescent dye, and display the intensity of fluorescence after each cycle. Devices comprising a thermal cycler, light beam emitter, and a fluorescent signal detector, have been described, e.g., in U.S. Pat. Nos. 5,928,907; 6,015,674; 6,174,670; and 6,814,934 and include, but are not limited to, the ABI Prism® 7700 Sequence Detection System (Applied Biosystems, Foster City, Calif.), the ABI GeneAmp® 5700 Sequence Detection System (Applied Biosystems, Foster City, Calif.), the ABI GeneAmp® 7300 Sequence Detection System (Applied Biosystems, Foster City, Calif.), the ABI GeneAmp® 7500 Sequence Detection System (Applied Biosystems, Foster City, Calif.), the StepOne™ Real-Time PCR System (Applied Biosystems, Foster City, Calif.) and/or the ABI GeneAmp® 7900 Sequence Detection System (Applied Biosystems, Foster City, Calif.). The oligonucleotides and/or oligonucleotide pairs used as primers in these methods is typically include at least one “allele-specific primer” that hybridizes to a sequence comprising the particular HLA allele to be amplified (e.g., the 4-digit HLA-B*51 subtype (e.g., HLA-B*51:01, HLA-B*51:02, HLA-B*51:03, HLA-B*51:05, HLA-B*51:07, HLA-B*51:08, HLA-B*51:09, HLA-B*51:10, HLA-B*51:11N, HLA-B*51:12, HLA-B*51:13, HLA-B*51:14, HLA-B*51:15, HLA-B*51:21, HLA-B*51:22, HLA-B*51:26, HLA-B*51:27N, HLA-B*51:29, HLA-B*51:31, HLA-B*51:32, HLA-B*51:33, HLA-B*51:36, HLA-B*51:37, HLA-B*51:63 and/or HLA-B*51:65 (e.g., any of SEQ ID NOS. 1-25)), and which when used in PCR can be extended to effectuate first strand cDNA synthesis. Allele-specific primers are specific for a particular allele of a given target DNA or loci and can be designed to detect a difference of as little as one nucleotide in the target sequence. Any method known to those of ordinary skill in the art may be used to measure the amplification of nucleic acids using the methods described herein.

The oligonucleotides used in the methods described herein (e.g., as primers and/or probes) may be partially or completely complementary to the target sequence of interest (e.g., the HLA-B*51 subtype sequence or HLA-B*57:01 sequence (e.g., as mRNA and/or cDNA). For instance, an oligonucleotide may be about 75-85%, 85-95%, 95-99% or 100% complementary to the target sequence of interest. The target-specific portion of the oligonucleotide may be about any of 4-30 nucleotides in length, such as about any of 4-10, 11-15, 16-20, 21-25, or 25-30 nucleotides in length. In some embodiments, the Tm of the target specific portion is about 5° C. below the anneal/extend temperature used for PCR cycling. In some embodiments, the Tm of the target specific portion of the allele-specific primer ranges from about any of 50° C. to 70° C., such as about any of 50° C. to 55° C., 55° C. to 60° C., 60° C. to 65° C., or 65° C. to 70° C. Such oligonucleotides are only meant to be exemplary as other types of oligonucleotides may also be suitable, as would be understood by those of ordinary skill in the art.

As mentioned herein, the oligonucleotide probe(s) may comprise (or come into contact with) one or more detectable labels that is/are typically attached thereto. The detectable label may emit a signal when free or when bound to one the target nucleic acid. The detectable label may also emit a signal when in proximity to another detectable label. Detectable labels may also be used with quencher molecules such that the signal is only detectable when not in sufficiently close proximity to the quencher molecule. In some embodiments, the detectable label may be attached to a probe, which may be incorporated into a primer, or may otherwise bind to amplified target nucleic acid (e.g., a detectable nucleic acid binding agent such as an intercalating or non-intercalating dye). When using more than one detectable label, each should differ in their spectral properties such that the labels may be distinguished from each other, or such that together the detectable labels emit a signal that is not emitted by either detectable label alone. Exemplary detectable labels include, for instance, a fluorescent dye or fluorphore (e.g., a chemical group that can be excited by light to emit fluorescence or phosphorescence), “acceptor dyes” capable of quenching a fluorescent signal from a fluorescent donor dye, and the like. Suitable detectable labels may include, for example, flurosceins (e.g., 5-carboxy-2,7-dichlorofluorescein; 5-Carboxyfluorescein (5-FAM); 5-HAT (Hydroxy Tryptamine); 5-Hydroxy Tryptamine (HAT); 6-JOE; 6-carboxyfluorescein (6-FAM); FITC); Alexa fluors (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE), coumarins (e.g., 7-amino-4-methylcoumarin, AMC, AMCA, AMCA-S, AMCA-X, ABQ, CPM methylcoumarin, coumarin phalloidin, hydroxycoumarin, CMFDA, methoxycoumarin), calcein, calcein AM, calcein blue, calcium dyes (e.g., calcium crimson, calcium green, calcium orange, calcofluor white), Cascade Blue, Cascade Yellow; Cy™ dyes (e.g., 3, 3.18, 3.5, 5, 5.18, 5.5, 7), cyan GFP, cyclic AMP Fluorosensor (FiCRhR), fluorescent proteins (e.g., green fluorescent protein (e.g., GFP. EGFP), blue fluorescent protein (e.g., BFP, EBFP, EBFP2, Azurite, mKalamal), cyan fluorescent protein (e.g., ECFP, Cerulean, CyPet), yellow fluorescent protein (e.g., YFP, Citrine, Venus, YPet), FRET donor/acceptor pairs (e.g., fluorescein/tetramethylrhodamine, IAEDANS/fluorescein, EDANS/dabcyl, fluorescein/fluorescein, BODIPY FL/BODIPY FL, Fluorescein/QSY7 and QSY9), LysoTracker and LysoSensor (e.g., LysoTracker Blue DND-22, LysoTracker Blue-White DPX, LysoTracker Yellow HCK-123, LysoTracker Green DND-26, LysoTracker Red DND-99, LysoSensor Blue DND-167, LysoSensor Green DND-189, LysoSensor Green DND-153, LysoSensor Yellow/Blue DND-160, LysoSensor Yellow/Blue 10,000 MW dextran), Oregon Green (e.g., 488, 488-X, 500, 514); rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, 5-ROX (carboxy-X-rhodamine), Sulphorhodamine B can C, Sulphorhodamine G Extra, Tetramethylrhodamine (TRITC), WT), Texas Red, Texas Red-X, VIC and other labels described in, e.g., U.S. Pub. No. 2009/0197254), among others as would be known to those of skill in the art.

Compositions comprising oligonucleotides and/or other reagents for use in identifying and/or quantitating the HLA alleles in a nucleic acid sample are also provided. For instance, such compositions may comprise one or more an allele-specific oligonucleotide primers and/or probes. In some embodiments, the compositions may further comprise one or more polymerases, dNTPs, reagents and/or buffers suitable for PCR amplification, and/or a template sequence or nucleic acid sample. In some embodiments, the one or more polymerases may be thermostable. Compositions may also comprise one or more control oligonucleotide primers and/or probes. Exemplary oligonucleotides that may be included in such compositions are any one or more of SEQ ID NOS. 37, 38, 39, 40, 41, 42, 43, 44 and/or 45. Compositions for use in amplifying and/or detecting any one or more HLA-B*51 subtypes of interest may comprise SEQ ID NOS. 40, 41 and/or 42, for instance. Compositions for use in amplifying and/or detecting HLA-B*57:01 may comprise SEQ ID NOS. 43, 44 and/or 45, for instance. Compositions for use in amplifying and/or detecting control sequences (e.g., human ribonuclease 30 kDa (RPP30)) may comprise SEQ ID NOS. 37, 38 and/or 39, for example. Such compositions may be used to amplify and/or detect any one or more HLA-B*51 subtypes of interest, HLA-B*57:01 and/or a control amplicon (e.g., human ribonuclease 30 kDa (RPP30)). These compositions may be provided alone (e.g., in a container), with one another (e.g., in the same and/or different containers), and/or as part of a kit as described herein.

Also provided herein are kits for identifying the presence of HLA-B*51 and/or HLA-B*57 alleles and/or subtypes in a biological sample. The kit typically comprises one or more oligonucleotides corresponding to one or more HLA-B*51 and/or HLA-B*57 alleles. The oligonucleotides may be labeled with one or more detectable labels. The one or more detectable labels may be, for example, a fluorescent or chromogenic label or a binding moiety such as biotin. These reagents may be free in solution or may be immobilized on a solid support, such as a magnetic bead, tube, microplate well, or chip. The kit may further comprise detection reagents such as a substrate, for example a chromogenic, fluorescent or chemiluminescent substrate, which reacts with the label, or with molecules, such as enzyme conjugates, which bind to the label, to produce a signal, and/or reagents for immunoprecipitation (i.e., protein A or protein G reagents). The detection reagents may further comprise buffer solutions, wash solutions, and other useful reagents. The reagents may be provided in one or more suitable containers (e.g., a vial) in which the contents are protected from the external environment. The kit may also comprise one or both of an apparatus for handling and/or storing the sample obtained from the individual and an apparatus for obtaining the sample from the individual (i.e., a needle, lancet, and collection tube or vessel). Where the assay is to be combined with another type of assay (e.g., a serologically-based assay), the required reagents for each of such assays (i.e., primers, buffers and the like) may also be included. Instructions for the use of such kits may also be included. Other types of kits may also be provided, as would be understood by one of ordinary skill in the art.

Thus, this disclosure provides methods for diagnosing of patients having Behçet's disease and/or being at risk of a hypersensitivity reaction to abacavir by determining the presence or absence of one or more HLA alleles by amplifying and detecting one or more HLA alleles using a real-time polymerase chain reaction (PCR). These methods are carried out by specifically amplifying and detecting the presence of alleles corresponding to either condition without amplifying alleles unrelated to such conditions.

For instance, in embodiments relating to diagnosing patients having Behçet's disease, the HLA allele may be HLA-B*51. In some embodiments, the amplifying is performed using one or more oligonucleotides having binding specificity for HLA-B*51. In certain embodiments, the method may comprise amplifying from a biological sample of an individual a nucleic acid molecule representing at least a portion of exon 2 and/or exon 3 a HLA-B*51 allele and detecting amplification of the nucleic acid wherein amplification of the nucleic acid indicates that the individual expresses an HLA-B*51 allele. Typically, these methods are carried out using real-time polymerase chain reaction (PCR) with forward and reverse primers having specificity for one or more HLA-B*51 alleles. In some embodiments, the methods provide for the detection of all 4-digit HLA-B*51 subtypes (e.g., HLA-B*51:01, HLA-B*51:02, HLA-B*51:03, HLA-B*51:05, HLA-B*51:07, HLA-B*51:08, HLA-B*51:09, HLA-B*51:10, HLA-B*51:11N, HLA-B*51:12, HLA-B*51:13, HLA-B*51:14, HLA-B*51:15, HLA-B*51:21, HLA-B*51:22, HLA-B*51:26, HLA-B*51:27N, HLA-B*51:29, HLA-B*51:31, HLA-B*51:32, HLA-B*51:33, HLA-B*51:36, HLA-B*51:37, HLA-B*51:63 and/or HLA-B*51:65 (e.g., any of SEQ ID NOS. 1-25)). In preferred embodiments, amplification of other HLA-B subtypes does not occur. In some embodiments, the one or more oligonucleotides having binding specificity for HLA-B*51 (e.g., any of the above-described HLA-B*51 subtypes) may be SEQ ID NO.:40 (e.g., as a forward primer) and/or SEQ ID NO.:41 (e.g., as a reverse primer). Some embodiments may also comprise hybridizing the amplified nucleic acid molecules to one or more nucleic acid probes specifically hybridizing to target regions within the nucleic acid molecules, the target regions comprising one or more polymorphic nucleotide sequences in exon 2 and/or 3 of HLA-B*51. In some embodiments, the amplified nucleic acid may correspond to any of HLA-B*51:01, HLA-B*51:02, HLA-B*51:03, HLA-B*51:05, HLA-B*51:07, HLA-B*51:08, HLA-B*51:09, HLA-B*51:10, HLA-B*51:11N, HLA-B*51:12, HLA-B*51:13, HLA-B*51:14, HLA-B*51:15, HLA-B*51:21, HLA-B*51:22, HLA-B*51:26, HLA-B*51:27N, HLA-B*51:29, HLA-B*51:31, HLA-B*51:32, HLA-B*51:33, HLA-B*51:36, HLA-B*51:37, HLA-B*51:63 and/or HLA-B*51:65 (e.g., any of SEQ ID NOS. 1-25), and/or a fragment and/or derivative thereof. In some embodiments, the probe may be or comprise SEQ ID NO.:42.

In embodiments relating to the identification of individuals at risk of a hypersensitivity reaction to abacavir, the HLA allele may be HLA-B*57:01 (e.g., a subtype of HLA-B*57). In such embodiments, the amplifying may be performed using one or more oligonucleotides having binding specificity for HLA-B*57:01. In certain embodiments, the method may comprise amplifying from a biological sample of an individual a nucleic acid molecule representing at least a portion of exon 3 HLA-B*57:01 and detecting amplification of the nucleic acid wherein amplification of the nucleic acid indicates that the individual expresses HLA-B*57:01. Typically, these methods are carried out using real-time polymerase chain reaction (PCR) with forward and reverse primers having specificity for HLA-B*57:01. In preferred embodiments, amplification of other HLA-B*57 subtypes does not occur. In some embodiments, the one or more oligonucleotides having binding specificity for HLA-B*57:01 may be SEQ ID NO.:43 (e.g., as a forward primer) and/or SEQ ID NO.:44 (e.g., as a reverse primer). Some embodiments may also comprise hybridizing the amplified nucleic acid molecules to one or more nucleic acid probes specifically hybridizing to target regions within the nucleic acid molecules, the target regions comprising one or more polymorphic nucleotide sequences in exon 3 of HLA-B*57:01. In some embodiments, the amplified nucleic acid may correspond to any of HLA-B*57:01, HLA-B*57:01:02, HLA-B*57:01:03, HLA-B*57:01:04, HLA-B*57:01:05, HLA-B*57:01; 06, HLA-B*57:01:07, HLA-B*57:01:08, HLA-B*57:01:09, HLA-B*57:01:10, and HLA-B*57:01:11 (e.g., SEQ ID NOS. 26-36, FIG. 2), and/or a fragment and/or derivative thereof. In some embodiments, the probe may be or comprise SEQ ID NO.:45.

In certain embodiments, the one or more nucleic acid probes may be labeled with one or more detectable labels. The real-time PCR assays may be single and/or multiplex assays. The methods may also comprise the amplification of one or more control nucleic acid sequences. For example, control nucleic acid sequences may be used to amplify and detect a control human DNA target (ribonuclease 30 kDa (RPP30)) using, for example, SEQ ID NO.:37 (e.g., as a forward primer), SEQ ID NO.:38 (e.g., as a reverse primer) and/or SEQ ID NO.:39 (e.g., as an oligonucleotide probe). As described above, kits are also provided. Such kits may include, for instance, oligonucleotides having the sequence of one or more of SEQ ID NOS. 40, 41 and/or 42 for amplifying and/or detecting HLA-B*51 alleles (e.g., any of the above-described subtypes) and/or one or more of SEQ ID NOS. 43, 44 and/or 45 for amplifying and/or detecting HLA-B*57:01. The kits may also include one or more control samples and/or control oligonucleotides (e.g., one or more of SEQ ID NOS. 37, 38 and/or 39). The oligonucleotides (e.g., the oligonucleotide probes) may also comprise a detectable label. The kits may further comprise instructions for use in amplifying and/or detecting HLA-B*51 alleles (e.g., any of the above-described subtypes) and/or HLA-B*57:01.

Throughout this disclosure, exemplification and/or definition of specific terms should be considered non-limiting. For example, the singular forms “a”, “an” and “the” include the plural unless the context clearly dictates otherwise. Approximating language, as used herein throughout the specification and claims, may be applied to modify any quantitative representation that could permissibly vary without resulting in a change in the basic function to which it is related. Accordingly, a value modified by a term such as “about” is not to be limited to the precise value specified. Where necessary, ranges have been supplied, and those ranges are inclusive of all sub-ranges there between. The use of the singular may include the plural unless specifically stated otherwise or unless, as will be understood by one of skill in the art in light of the present disclosure, the singular is the only functional embodiment. Thus, for example, “a” may mean more than one, and “one embodiment” may mean that the description applies to multiple embodiments. The phrase “and/or” denotes a shorthand way of indicating that the specific combination is contemplated in combination and, separately, in the alternative.

It will be appreciated that there is an implied “about” prior to the temperatures, concentrations, times, etc. discussed in the present teachings, such that slight and insubstantial deviations are within the scope of the present teachings herein. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the invention.

Unless specifically noted herein, embodiments in the above specification that recite “comprising” various components may also be contemplated as “consisting of” or “consisting essentially of” the recited components; embodiments in the specification that recite “consisting of” various components may also be contemplated as “comprising” or “consisting essentially of” the recited components; and embodiments in the specification that recite “consisting essentially of” various components may also be contemplated as “consisting of” or “comprising” the recited components.

### EXAMPLES

### Example 1

### Assay System for Identifying HLA B*51

Behçet's disease is a rare immune-mediated small-vessel systemic vasculitis that often presents with mucous membrane ulceration and ocular problems. As a systemic disease, it can also involve visceral organs such as the gastrointestinal tract, pulmonary, musculoskeletal, cardiovascular and neurological systems. This syndrome can be fatal due to ruptured vascular aneurysms or severe neurological complications. A large number of serological studies show a linkage between the disease and HLA-B*51. The presence of the HLA-B*51 in a patient is an indication or a confirmation of Behçet's disease diagnosis. Currently, most laboratories use a 2-digit test (e.g., Luminex) to identify the HLA-B*51 haplotype.

The real-time PCR systems described herein provide for the amplification and/or detection of all 4 digit HLA-B*51 subtypes (specifically, HLA-B*51:01, HLA-B*51:02, HLA-B*51:03, HLA-B*51:05, HLA-B*51:07, HLA-B*51:08, HLA-B*51:09, HLA-B*51:10, HLA-B*51:11N, HLA-B*51:12, HLA-B*51:13, HLA-B*51:14, HLA-B*51:15, HLA-B*51:21, HLA-B*51:22, HLA-B*51:26, HLA-B*51:27N, HLA-B*51:29, HLA-B*51:31, HLA-B*51:32, HLA-B*51:33, HLA-B*51:36, HLA-B*51:37, HLA-B*51:63 and HLA-B*51:65) to the exclusion of other (e.g., all other) HLA-B subtypes. Exons 2 and 3 were selected for amplification because the differences between HLA-B*51 and others close alleles in those exons allows for the discrimination between the same. The DNA primers used in these assays are shown Tables 1 and 2 as well as FIG. 1 (HLA-B*51 primers and probe). Duplex PCR using a control human DNA target (ribonuclease 30 kDa (RPP30)) was amplified in these assays as an internal control (Table 2).

TABLE 1
B*51 Primers and Probe

| | 5′ | Inner | 3′ | |
|---|---|---|---|---|
| Primer | Modification | Modification | Modification | Sequence (5′-3′) |
| B51_F | None | None | None | GAG TAT TGG GAC CGG AAC |
| (forward | | | | ACA (SEQ ID NO.: 40) |
| primer) | | | | |
| B51_R | None | None | None | AGC CAT ACA TCG TCT GCC |
| (reverse | | | | AAG T (SEQ ID NO.: 41) |
| primer) | | | | |
| B51_P | FAM | None | BHQ-1 | AGA ACC TGC GGA TCG CGC |
| (probe) | (Fluorescein) | | | TCC (SEQ ID NO.: 42) |

TABLE 2
Control(RPP30) Primers and Probe

| | 5′ | Inner | 3′ | |
|---|---|---|---|---|
| Primer | Modification | Modification | Modification | Sequence (5′-3′) |
| HDNA_F | None | None | None | CAG ATT TGG ACC TGC |
| (forward | | | | GAG CG |
| primer) | | | | (SEQ ID NO.: 37) |
| HDNA_R | None | None | None | AGG TGA GCG GCT GTC |
| (reverse | | | | TCC ACA |
| primer) | | | | (SEQ ID NO.: 38) |
| HDNA_P | Yakima | None | BHQ-1 | TTC TGA CCT GAA GGC |
| (probe) | Yellow | | | TCT GCG CGG |
| | | | | (SEQ ID NO.: 39) |

The specificity against the most frequent HLA-B genotypes (B*07, 08, 13, 14, 15, 18, 27, 35, 37, 38, 39, 40, 41, 42, 44, 45 47, 49, 50, 52, 53, 54, 55, 56, 57, 58, 67, 81) in Switzerland has been tested. No cross-reaction has been observed in more than 100 samples with HLA-B haplotypes. One hundred (100) samples HLA-B identified and further characterized using PCR-SSO (Luminex) were tested blindly by IAL-HLA B*51 real-time PCR assay described herein. The resultant data is summarized in Table 3 and exemplary advantages of the system are summarized in Tables 4 and 5. As shown therein, the specificity of the IAL PCR assay for B*51 using the primers and probe of Table 1 was excellent.

TABLE 3
Experimental Results***

Luminex

| | | |
|---|---|---|
| HLA B*51 | Positive | Negative |

| | | | |
|---|---|---|---|
| Real-time PCR assay | Positive | 73 | 0 |
| using primers and | Negative | 0 | 27* |
| probe of Table 1 | | | |

***Other subtypes found in the 27 non-HLA-B*51 samples: B*07, 08, 13, 14, 15, 18, 27, 35, 37, 38, 39, 40, 41, 42, 44, 45 47, 49, 50, 52, 53, 54, 55, 56, 57, 58, 67, and 81.

TABLE 4
Reagent Cost Advantage ****

| | Current | Real-time |
|---|---|---|
| Steps | Cost | PCR Cost |
| DNA extraction | 12.95 | 12.95 |
| Amplification/Detection | 76.55 | 8.60 |
| Total | 89.50 | 21.55 |

**** Prices indicated in CHF

TABLE 5
Time Advantage (minutes)

| | | Real-time |
|---|---|---|
| Steps (in minutes) | Current | PCR |
| DNA extraction | 48 (28) | 38 (18) |
| Amplification/Detection | 315 (125) | 150 (30) |
| Total | 353 (153) | 188 (48) |

In summary, the IAL HLA-B*51 real-time PCR assay was determined to be highly specific and highly reproducible; to be simpler, faster and less expensive than existing assay systems (e.g., four-fold less expensive than other available assays); not to require expensive/large laboratory equipment; to provide a ready-to-implement point-of-care (“PoC”) assay system; and, to be suitable for rapid commercial development.

### Example 2

### Assay System for Identifying HLA B*5701

Carriage of the major histocompatibility complex class I allele HLA-B*57:01 has been linked with increase hypersensitivity reaction to the drug Abacavir. Abacavir is a nucleoside reverse-transcriptase inhibitor with activity against the human immunodeficiency virus (HIV), available for once-daily use in combination with other antiretroviral agents, that has shown efficacy, few drug interactions, and a favorable long-term toxicity profile. HLA-B*57:01 screening is indicated for new HIV patients, and must be performed before the use of Abacavir to prevent the life threatening hypersensitivity reaction to abacavir. Currently, most laboratories either identify HLA-B*57 alleles by sequencing or using two-digit test (i.e. Luminex) to identify the HLA-B*57 haplotype. The real-time PCR assay described herein provides for the identification of all subtypes of B*57:01 to 6 digits (e.g., B*57:01:01-B*57:01:11) and exclude other B*57 subtypes (B*57:02-B*57:44), particularly B*57:02/03. Exon 3 was selected for amplification because the differences therein allow accurate discrimination between the different HLA B*57 alleles. For example, few very rare genotypes (e.g., B*57:06, B*57:08 and/or B*57:10) are detected using this method. Duplex PCR using a control human DNA target (ribonuclease 30 kDa (RPP30)) was amplified in these assays as an internal control.

To design the B*57:01 primers, B*57:01:01 was used as a reference sequence and aligned with other B*57:01 subtypes (B*57:01:02, B*57:01:03, B*57:01:04, B*57:01:05, B*57:01; 06, B*57:01:07, B*57:01:08, B*57:01:09, B*57:01:10, and B*57:01:11). B*57:01:01 was also aligned with B57 subtypes that are not to be detected in these assays (B*57:02, B*57:03, B*57:04, B*57:05, B*57:06, B*57:07, B*57:08, B*57:09, and B*57:10). B*55:14 was also aligned with B*57:01:01 to exclude it from amplification which has been shown to be frequently unintentionally amplified by existing methods. The DNA primers and selected as described herein are shown in Table 6 and FIG. 2 (control primers and probe are shown in Table 2 above).

TABLE 6
B*57:01 Primers and Probe

| | 5′ | Inner | 3′ | |
|---|---|---|---|---|
| Primer | Modification | Modification | Modification | Sequence (5′-3′) |
| B5701_F | None | None | None | GTC TCA CAT CAT CCA |
| (forward | | | | GGT GAT GTA TG |
| primer) | | | | (SEQ ID NO.: 43) |
| B5701_R | None | None | None | GGT GAT CTG AGC CGC |
| (reverse | | | | CGT (SEQ ID NO.: 44) |
| primer) | | | | |
| B5701_P | FAM | None | BHQ-1 | CCT CCG CGG GCA TGA |
| (probe) | (Fluorescein) | | | CCA GTC (SEQ ID NO.: 45) |

The specificity of these primers/probes for the most frequent HLA-B genotypes (B*07, 08, 13, 14, 15, 18, 27, 35, 37, 38, 39, 40, 41, 44, 45, 49, 50, 51, 52, 53, 55, 58) was tested. No cross-reaction has been observed in more than 100 samples with HLA-B haplotypes. Ninety (90) samples identified as HLA-B*57 and further characterized using sequencing and 4-digit analysis were tested blindly by IAL HLA-B*57 real-time PCR. The resultant data is summarized in Table 7 and exemplary advantages of the system are summarized in Tables 8 and 9. As shown therein, the specificity of the IAL HLA-B*57 real-time PCR assay for B*57:01 was excellent.

TABLE 7
Experimental Results

Sequencing

| | | |
|---|---|---|
| HLA B*57:01 | Positive | Negative |

| | | | |
|---|---|---|---|
| Real-time PCR assay | Positive | 57 | 0 |
| using primers and | Negative | 0 | 33* |
| probe of Table 6 | | | |

*Other subtypes present in the 33 non-HLA-B*57:01 samples included B*57:02/03/04.

TABLE 8
Cost Advantage **

| | Current | Real-time |
|---|---|---|
| Steps | Cost | PCR Cost |
| DNA extraction | 12.95 | 12.95 |
| Amplification/Detection | 76.55 | 8.60 |
| Negative sample | 89.50 | 21.55 |
| Sequencing | 112.85 | — |
| Positive sample | 202.35 | 21.55 |

** Prices indicated in CHF

TABLE 9
Time Advantage (minutes)

| | | Real-time |
|---|---|---|
| Steps | Current | PCR |
| DNA extraction | 48 (28) | 38 (18) |
| Amplification/Detection | 315 (125) | 150 (30) |
| Negative sample | 353 (153) | 188 (48) |
| Sequencing | 620 (90) | — |
| Positive sample | 973 (243) | 188 (48) |

In summary, the IAL HLA-B*57:01 real-time PCR assay was determined to be highly specific and highly reproducible; to be simpler, faster and less expensive than existing assay systems (e.g., ten-fold less expensive than the retail price of COBAS® AmpliPrep/COBAS® TaqMan® HLA-B*5701 Screening Test of US$137.50); not to require expensive/large laboratory equipment; to provide a ready-to-implement point-of-care (“PoC”) assay system; and, to be suitable for rapid commercial development.

All references cited within this disclosure are hereby incorporated by reference in their entirety. While certain embodiments are described herein, such as in terms of the preferred embodiments, it is understood that variations and modifications will occur to those skilled in the art. Other embodiments of the methods and reagents described herein will be evident to the skilled artisan from this disclosure. Therefore, it is intended that the appended claims cover all such equivalent variations that come within the scope of the claims.

SEQUENCE LISTING

>imgthla|HLA00344|HLA00344 HLA-B*51:01:01, Human MHC Class I sequence
(SEQ ID NO: 1)
ATGCGGGTCACGGCGCCCCGAACCGTCCTCCTGCTGCTCTGGGGGGCAGTGGCCCTGACCGAGACCTGGGCCGGCTCCCA
CTCCATGAGGTATTTCTACACCGCCATGTCCCGGCCCGGCCGCGGGGAGCCCCGCTTCATTGCAGTGGGCTACGTGGACG
ACACCCAGTTCGTGAGGTTCGACAGCGACGCCGCGAGTCCGAGGACGGAGCCCCGGGCGCCATGGATAGAGCAGGAGGGG
CCGGAGTATTGGGACCGGAACACACAGATCTTCAAGACCAACACACAGACTTACCGAGAGAACCTGCGGATCGCGCTCCG
CTACTACAACCAGAGCGAGGCCGGGTCTCACACTTGGCAGACGATGTATGGCTGCGACGTGGGGCCGGACGGGCGCCTCC
TCCGCGGGCATAACCAGTACGCCTACGACGGCAAAGATTACATCGCCCTGAACGAGGACCTGAGCTCCTGGACCGCGGCG
GACACCGCGGCTCAGATCACCCAGCGCAAGTGGGAGGCGGCCCGTGAGGCGGAGCAGCTGAGAGCCTACCTGGAGGGCCT
GTGCGTGGAGTGGCTCCGCAGACACCTGGAGAACGGGAAGGAGACGCTGCAGCGCGCGGACCCCCCAAAGACACACGTGA
CCCACCACCCCGTCTCTGACCATGAGGCCACCCTGAGGTGCTGGGCCCTGGGCTTCTACCCTGCGGAGATCACACTGACC
TGGCAGCGGGATGGCGAGGACCAAACTCAGGACACTGAGCTTGTGGAGACCAGACCAGCAGGAGATAGAACCTTCCAGAA
GTGGGCAGCTGTGGTGGTGCCTTCTGGAGAAGAGCAGAGATACACATGCCATGTACAGCATGAGGGGCTGCCGAAGCCCC
TCACCCTGAGATGGGAGCCATCTTCCCAGTCCACCATCCCCATCGTGGGCATTGTTGCTGGCCTGGCTGTCCTAGCAGTT
GTGGTCATCGGAGCTGTGGTCGCTACTGTGATGTGTAGGAGGAAGAGCTCAGGTGGAAAAGGAGGGAGCTACTCTCAGGC
TGCGTCCAGCGACAGTGCCCAGGGCTCTGATGTGTCTCTCACAGCTTGA
>imgthla|HLA00347|HLA00347 HLA-B*51:02:02, Human MHC Class I sequence
(SEQ ID NO: 2)
ATGCGGGTCACGGCGCCCCGAACCGTCCTCCTGCTGCTCTGGGGGGCAGTGGCCCTGACCGAGACCTGGGCCGGCTCCCA
CTCCATGAGGTATTTCTACACCGCCATGTCCCGGCCCGGCCGCGGGGAGCCCCGCTTCATCGCAGTGGGCTACGTGGACG
ACACCCAGTTCGTGAGGTTCGACAGCGACGCCGCGAGTCCGAGGACGGAGCCCCGGGCGCCATGGATAGAGCAGGAGGGG
CCGGAGTATTGGGACCGGAACACACAGATCTTCAAGACCAACACACAGACTTACCGAGAGAACCTGCGGATCGCGCTCCG
CTACTACAACCAGAGCGAGGCCGGGTCTCACACTTGGCAGACGATGTATGGCTGCGACGTGGGGCCGGACGGGCGCCTCC
TCCGCGGGCATAACCAGTACGCCTACGACGGCAAAGATTACATCGCCCTGAACGAGGACCTGAGCTCCTGGACCGCGGCG
GACACCGCGGCTCAGATCACCCAGCGCAAGTGGGAGGCGGCCCGTGAGGCGGAGCAGCTGAGAGCCTACCTGGAGGGCCT
GTGCGTGGAGTGGCTCCGCAGATACCTGGAGAACGGGAAGGAGACGCTGCAGCGCGCGGACCCCCCAAAGACACACGTGA
CCCACCACCCCGTCTCTGACCATGAGGCCACCCTGAGGTGCTGGGCCCTGGGCTTCTACCCTGCGGAGATCACACTGACC
TGGCAGCGGGATGGCGAGGACCAAACTCAGGACACTGAGCTTGTGGAGACCAGACCAGCAGGAGATAGAACCTTCCAGAA
GTGGGCAGCTGTGGTGGTGCCTTCTGGAGAAGAGCAGAGATACACATGCCATGTACAGCATGAGGGGCTGCCGAAGCCCC
TCACCCTGAGATGGGAGCCATCTTCCCAGTCCACCATCCCCATCGTGGGCATTGTTGCTGGCCTGGCTGTCCTAGCAGTT
GTGGTCATCGGAGCTGTGGTCGCTACTGTGATGTGTAGGAGGAAGAGCTCAGGTGGAAAAGGAGGGAGCTACTCTCAGGC
TGCGTCCAGCGACAGTGCCCAGGGCTCTGATGTGTCTCTCACAGCTTGA
>imgthla|HLA00348|HLA00348 HLA-B*51:03, Human MHC Class I sequence
(SEQ ID NO: 3)
ATGCGGGTCACGGCGCCCCGAACCGTCCTCCTGCTGCTCTGGGGGGCAGTGGCCCTGACCGAGACCTGGGCCGGCTCCCA
CTCCATGAGGTATTTCTACACCGCCATGTCCCGGCCCGGCCGCGGGGAGCCCCGCTTCATTGCAGTGGGCTACGTGGACG
ACACCCAGTTCGTGAGGTTCGACAGCGACGCCGCGAGTCCGAGGACGGAGCCCCGGGCGCCATGGATAGAGCAGGAGGGG
CCGGAGTATTGGGACCGGAACACACAGATCTTCAAGACCAACACACAGACTTACCGAGAGAACCTGCGGATCGCGCTCCG
CTACTACAACCAGAGCGAGGCCGGGTCTCACACTTGGCAGACGATGTATGGCTGCGACGTGGGGCCGGACGGGCGCCTCC
TCCGCGGGCATAACCAGTACGCCTACGACGGCAAAGATTACATCGCCCTGAACGAGGACCTGAGCTCCTGGACCGCGGCG
GACACCGCGGCTCAGATCACCCAGCGCAAGTGGGAGGCGGCCCGTGAGGCGGAGCAGCTGAGAGCCTACCTGGAGGGCCT
GTGCGTGGAGGGGCTCCGCAGACACCTGGAGAACGGGAAGGAGACGCTGCAGCGCGCGGACCCCCCAAAGACACACGTGA
CCCACCACCCCGTCTCTGACCATGAGGCCACCCTGAGGTGCTGGGCCCTGGGCTTCTACCCTGCGGAGATCACACTGACC
TGGCAGCGGGATGGCGAGGACCAAACTCAGGACACTGAGCTTGTGGAGACCAGACCAGCAGGAGATAGAACCTTCCAGAA
GTGGGCAGCTGTGGTGGTGCCTTCTGGAGAAGAGCAGAGATACACATGCCATGTACAGCATGAGGGGCTGCCGAAG0000
TCACCCTGAGATGGGAGCCATCTTCCCAGTCCACCATCCCCATCGTGGGCATTGTTGCTGGCCTGGCTGTCCTAGCAGTT
GTGGTCATCGGAGCTGTGGTCGCTACTGTGATGTGTAGGAGGAAGAGCTCAGGTGGAAAAGGAGGGAGCTACTCTCAGGC
TGCGTCCAGCGACAGTGCCCAGGGCTCTGATGTGTCTCTCACAGCTTGA
>imgthla|HLA00350|HLA00350 HLA-B*51:05, Human MHC Class I sequence (partial)
(SEQ ID NO: 4)
GCTCCCACTCCATGAGGTATTTCTACACCGCCATGTCCCGGCCCGGCCGCGGGGAGCCCCGCTTCATCGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGTGGCGGAGCAGCGGAGAGCCTACCTGG
AGGGCCTGTGCGTGGAGTGGCTCCGCAGATACCTGGAGAACGGGAAGGAGACGCTGCAGCGCGCGGACCCCCCAAAGACA
CACGTGACCCACCACCCCGTCTCTGACCATGAGGCCACCCTGAGGTGCTGGGCCCTGGGCTTCTACCCTGCGGAGATCAC
ACTGACCTGGCAGCGGGATGGCGAGGACCAAACTCAGGACACTGAGCTTGTGGAGACCAGACCAGCAGGAGATAGAACCT
TCCAGAAGTGGGCAGCTGTGGTGGTGCCTTCTGGAGAAGAGCAGAGATACACATGCCATGTACAGCATGAGGGGCTGCCG
AAGCCCCTCACCCTGAGATGGG
>imgthla|HLA00352|HLA00352 HLA-B*51:07:01, Human MHC Class I sequence
(SEQ ID NO: 5)
ATGCGGGTCACGGCGCCCCGAACCGTCCTCCTGCTGCTCTGGGGGGCAGTGGCCCTGACCGAGACCTGGGCCGGCTCCCA
CTCCATGAGGTATTTCTACACCGCCATGTCCCGGCCCGGCCGCGGGGAGCCCCGCTTCATTGCAGTGGGCTACGTGGACG
ACACCCAGTTCGTGAGGTTCGACAGCGACGCCGCGAGTCCGAGGACGGAGCCCCGGGCGCCATGGATAGAGCAGGAGGGG
CCGGAGTATTGGGACCGGAACACACAGATCTCCAAGACCAACACACAGACTTACCGAGAGAACCTGCGGATCGCGCTCCG
CTACTACAACCAGAGCGAGGCCGGGTCTCACACTTGGCAGACGATGTATGGCTGCGACGTGGGGCCGGACGGGCGCCTCC
TCCGCGGGCATAACCAGTACGCCTACGACGGCAAAGATTACATCGCCCTGAACGAGGACCTGAGCTCCTGGACCGCGGCG
GACACCGCGGCTCAGATCACCCAGCGCAAGTGGGAGGCGGCCCGTGAGGCGGAGCAGCTGAGAGCCTACCTGGAGGGCCT
GTGCGTGGAGTGGCTCCGCAGACACCTGGAGAACGGGAAGGAGACGCTGCAGCGCGCGGACCCCCCAAAGACACACGTGA
CCCACCACCCCGTCTCTGACCATGAGGCCACCCTGAGGTGCTGGGCCCTGGGCTTCTACCCTGCGGAGATCACACTGACC
TGGCAGCGGGATGGCGAGGACCAAACTCAGGACACTGAGCTTGTGGAGACCAGACCAGCAGGAGATAGAACCTTCCAGAA
GTGGGCAGCTGTGGTGGTGCCTTCTGGAGAAGAGCAGAGATACACATGCCATGTACAGCATGAGGGGCTGCCGAAG0000
TCACCCTGAGATGGGAGCCATCTTCCCAGTCCACCATCCCCATCGTGGGCATTGTTGCTGGCCTGGCTGTCCTAGCAGTT
GTGGTCATCGGAGCTGTGGTCGCTACTGTGATGTGTAGGAGGAAGAGCTCAGGTGGAAAAGGAGGGAGCTACTCTCAGGC
TGCGTCCAGCGACAGTGCCCAGGGCTCTGATGTGTCTCTCACAGCTTGA
>imgthla|HLA00353|HLA00353 HLA-B*51:08, Human MHC Class I sequence
(SEQ ID NO: 6)
ATGCGGGTCACGGCGCCCCGAACCGTCCTCCTGCTGCTCTGGGGGGCAGTGGCCCTGACCGAGACCTGGGCCGGCTCCCA
CTCCATGAGGTATTTCTACACCGCCATGTCCCGGCCCGGCCGCGGGGAGCCCCGCTTCATTGCAGTGGGCTACGTGGACG
ACACCCAGTTCGTGAGGTTCGACAGCGACGCCGCGAGTCCGAGGACGGAGCCCCGGGCGCCATGGATAGAGCAGGAGGGG
CCGGAGTATTGGGACCGGAACACACAGATCTTCAAGACCAACACACAGACTTACCGAGAGAACCTGCGGATCGCGCTCCG
CTACTACAACCAGAGCGAGGCCGGGTCTCACACTTGGCAGACGATGTATGGCTGCGACGTGGGGCCGGACGGGCGCCTCC
TCCGCGGGCATAACCAGTACGCCTACGACGGCAAAGATTACATCGCCCTGAACGAGGACCTGAGCTCCTGGACCGCGGCG
GACACCGCGGCTCAGATCACCCAGCGCAAGTGGGAGGCGGCCCGTGTGGCGGAGCAGGACAGAGCCTACCTGGAGGGCCT
GTGCGTGGAGTGGCTCCGCAGACACCTGGAGAACGGGAAGGAGACGCTGCAGCGCGCGGACCCCCCAAAGACACACGTGA
CCCACCACCCCGTCTCTGACCATGAGGCCACCCTGAGGTGCTGGGCCCTGGGCTTCTACCCTGCGGAGATCACACTGACC
TGGCAGCGGGATGGCGAGGACCAAACTCAGGACACTGAGCTTGTGGAGACCAGACCAGCAGGAGATAGAACCTTCCAGAA
GTGGGCAGCTGTGGTGGTGCCTTCTGGAGAAGAGCAGAGATACACATGCCATGTACAGCATGAGGGGCTGCCGAAG0000
TCACCCTGAGATGGGAGCCATCTTCCCAGTCCACCATCCCCATCGTGGGCATTGTTGCTGGCCTGGCTGTCCTAGCAGTT
GTGGTCATCGGAGCTGTGGTCGCTACTGTGATGTGTAGGAGGAAGAGCTCAGGTGGAAAAGGAGGGAGCTACTCTCAGGC
TGCGTCCAGCGACAGTGCCCAGGGCTCTGATGTGTCTCTCACAGCTTGA
>imgthla|HLA00354|HLA00354 HLA-B*51:09:01, Human MHC Class I sequence (partial)
(SEQ ID NO: 7)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGTGGCGGAGCAGCTGAGAGCCTACCTGG
AGGGCCTGTGCGTGGAGTGGCTCCGCAGACACCTGGAGAACGGGAAGGAGACGCTGCAGCGCGCGG
>imgthla|HLA00355|HLA00355 HLA-B*51:10, Human MHC Class I sequence (partial)
(SEQ ID NO: 8)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAGGATTACATCGCCCTGAACGAGGACCTGCGCTCCTGGAC
CGCCGCGGACACGGCGGCTCAGATCACCCAGCGCAAGTGGGAGGCGGCCCGTGTGGCGGAGCAGCTGAGAGCCTACCTGG
AGGGCGAGTGCGTGGAGTGGCTCCGCAGATACCTGGAGAACGGGAAGGAGACGCTGCAGCGCGCGGACCCCCCAAAGACA
CACGTGACCCACCACCCCGTCTCTGACCATGAGGCCACCCTGAGGTGCTGGGCCCTGGGCTTCTACCCTGCGGAGATCAC
ACTGACCTGGCAGCGGGATGGCGAGGACCAAACTCAGGACACTGAGCTTGTGGAGACCAG
>imgthla|HLA00356|HLA00356 HLA-B*51:11N, Human MHC Class I sequence (partial)
(SEQ ID NO: 9)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGGACCCCCCCAAAGAC
ACACGTGACCCACCACCCCGTCTCTGACCATGAGGCCACCCTGAGGTGCTGGGCCCTGGGCTTCTACCCTGCGGAGATCA
CACTGACCTGGCAGCGGGATGGCGAGGACCAAACTCAGGACACTGAGCTTGTGGAGACCAGACCAGCAGGAGATAGAACC
TTCCAGAAGTGGGCAGCTGTGGTGGTGCCTTCTGGAGAAGAGCAGAGATACACATGCCATGTACAGCATGAGGGGCTGCC
GAAGCCCCTCACCCTGAGATGGG
>imgthla|HLA00357|HLA00357 HLA-B*51:12, Human MHC Class I sequence (partial)
(SEQ ID NO: 10)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCGACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGG
>imgthla|HLA00358|HLA00358 HLA-B*51:13:01, Human MHC Class I sequence (partial)
(SEQ ID NO: 11)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTTCGCCTACGACGGCAAGGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGG
>imgthla|HLA00359|HLA00359 HLA-B*51:14, Human MHC Class I sequence (partial)
(SEQ ID NO: 12)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAAACAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGG
>imgthla|HLA00360|HLA00360 HLA-B*51:15, Human MHC Class I sequence (partial)
(SEQ ID NO: 13)
GCTCCCACTCCATGAGGTATTTCTACACCGCCATGTCCCGGCCCGGCCGCGGGGAGCCCCGCTTCATCGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACCTGGGGCCGGACGGG
CGCCTCCTCCGCGGGCATAACCAGTTAGCCTACGACGGCAAGGATTACATCGCCCTGAACGAGGACCTGAGCTCCTGGAC
CGCGGCGGACACCGCGGCTCAGATCACCCAGCGCAAGTGGGAGGCGGCCCGTGTGGCGGAGCAGCTGAGAGCCTACCTGG
AGGGCCTGTGCGTGGAGTGGCTCCGCAGATACCTGGAGAACGGGAAGGAGACGCTGCAGCGCGCGG
>imgthla|HLA01134|HLA01134 HLA-B*51:21, Human MHC Class I sequence (partial)
(SEQ ID NO: 14)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCACGTGCGTGGAGTGGCTCCGCAGACACCTGGAGAACGGGAAGGAGACGCTGCAGCGCGCGG
>imgthla|HLA01243|HLA01243 HLA-B*51:22, Human MHC Class I sequence (partial)
(SEQ ID NO: 15)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTGCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGG
>imgthla|HLA01417|HLA01417 HLA-B*51:26, Human MHC Class I sequence (partial)
(SEQ ID NO: 16)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGACCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGG
>imgthla|HLA01474|HLA01474 HLA-B*51:27N, Human MHC Class I sequence (partial)
(SEQ ID NO: 17)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCTAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGG
>imgthla|HLA01568|HLA01568 HLA-B*51:29, Human MHC Class I sequence (partial)
(SEQ ID NO: 18)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCGGAGAGCCTACCTGG
AGGGCCTGTGCGTGGAGTGGCTCCGCAGACACCTGGAGAACGGGAAGGAGACGCTGCAGCGCGCGGACCCCCCAAAGACA
CACGTGACCCACCACCCCGTCTCTGACCATGAGGCCACCCTGAGGTGCTGGGCCCTGGGCTTCTACCCTGCGGAGATCAC
ACTGACCTGGCAGCGGGATGGCGAGGACCAAACTCAGGACACTGAGCTTGTGGAGACCAGACCAGCAGGAGATAGAACCT
TCCAGAAGTGGGCAGCTGTGGTGGTGCCTTCTGGAGAAGAGCAGAGATACACATGCCATGTACAGCATGAGGGGCTGCCG
AAGCCCCTCACCCTGAGATGGG
>imgthla|HLA01626|HLA01626 HLA-B*51:31, Human MHC Class I sequence (partial)
(SEQ ID NO: 19)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGTGGCGGAGCAGCTGAGAGCCTACCTGG
AGGGCGAGTGCGTGGAGTGGCTCCGCAGACACCTGGAGAACGGGAAGGAGACGCTGCAGCGCGCGG
>imgthla|HLA01657|HLA01657 HLA-B*51:32, Human MHC Class I sequence (partial)
(SEQ ID NO: 20)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGGACCCCCCAAAGACA
CACGTGACCCACCACCCCGTCTCTGACCATGAGGCCACCCTGAGGTGCTGGGCCCTGGGCTTCTACCCTGCGGAGATCAC
ACTGACCTGGCAGCGGGATGGCGAGGACCAAACTCAGGACACTGAGCTTGTGGAGACCAGACCAGCAGGAGATAGAACCT
TCCAGAAGTGGGCAGCTGTGGTGGTGCCTTCTGGAGAAGAGCAGAGATACACATGCCATGTACAGCATGAGGGGCTGCTG
AAGCCCCTCACCCTGAGATGGG
>imgthla|HLA01676|HLA01676 HLA-B*51:33, Human MHC Class I sequence (partial)
(SEQ ID NO: 21)
GCTCCCACTTCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGG
>imgthla|HLA02003|HLA02003 HLA-B*51:36, Human MHC Class I sequence (partial)
(SEQ ID NO: 22)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGGACAGAGCCTACCTGG
AGGGCACGTGCGTGGAGTGGCTCCGCAGATACCTGGAGAACGGGAAGGAGACGCTGCAGCGCGCGGACCCCCCAAAGACA
CACGTGACCCACCACCCCGTCTCTGACCATGAGGCCACCCTGAGGTGCTGGGCCCTGGGCTTCTACCCTGCGGAGATCAC
ACTGACCTGGCAGCGGGATGGCGAGGACCAAACTCAGGACACTGAGCTTGTGGAGACCAGACCAGCAGGAGATAGAACCT
TCCAGAAGTGGGCAGCTGTGGTGGTGCCTTCTGGAGAAGAGCAGAGATACACATGCCATGTACAGCATGAGGGGCTGCCG
AAGCCCCTCACCCTGAGATGGG
>imgthla|HLA02020|HLA02020 HLA-B*51:37, Human MHC Class I sequence (partial)
(SEQ ID NO: 23)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATGACCAGTCCGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGG
>imgthla|HLA03509|HLA03509 HLA-B*51:63, Human MHC Class I sequence (partial)
(SEQ ID NO: 24)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGGGCCGGACGGG
CGCCTCCTCCGCGGGCATAACCAGTCCGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGG
>imgthla|HLA03617|HLA03617 HLA-B*51:65, Human MHC Class I sequence (partial)
(SEQ ID NO: 25)
GCTCCCACTCCATGAGGTATTTCTACACCGCCATGTCCCGGCCCGGCCGCGGGGAGCCCCGCTTCATTGCAGTGGGCTAC
GTGGACGACACCCAGTTCGTGAGGTTCGACAGCGACGCCGCGAGTCCGAGGACGGAGCCCCGGGCGCCATGGATAGAGCA
GGAGGGGCCGGAGTATTGGGACCGGAACACACAGATCTTCAAGACCAACACACAGACTTACCGAGAGAACCTGCGGATCG
CGCTCCGCTACTACAACCAGAGCGAGGCCGGGTCTCACACTTGGCAGACGATGTATGGCTGCGACGTGGAGCCGGACGGG
CGCCTCCTCCGCGGGCATAACCAGTACGCCTACGACGGCAAAGATTACATCGCCCTGAACGAGGACCTGAGCTCCTGGAC
CGCGGCGGACACCGCGGCTCAGATCACCCAGCGCAAGTGGGAGGCGGCCCGTGAGGCGGAGCAGCTGAGAGCCTACCTGG
AGGGCCTGTGCGTGGAGTGGCTCCGCAGACACCTGGAGAACGGGAAGGAGACGCTGCAGCGCGCGGACCCCCCAAAGACA
CACGTGACCCACCACCCCGTCTCTGACCATGAGGCCACCCTGAGGTGCTGGGCCCTGGGCTTCTACCCTGCGGAGATCAC
ACTGACCTGGCAGCGGGATGGCGAGGACCAAACTCAGGACACTGAGCTTGTGGAGACCAGACCAGCAGGAGATAGAACCT
TCCAGAAGTGGGCAGCTGTGGTGGTGCCTTCTGGAGAAGAGCAGAGATACACATGCCATGTACAGCATGAGGGGCTGCCG
AAGCCCCTCACCCTGAGATGG
>imgthla|HLA00381|HLA00381 HLA-B*57:01:01, Human MHC Class I sequence
(SEQ ID NO: 26)
ATGCGGGTCACGGCACCCCGAACCGTCCTCCTGCTGCTCTGGGGGGCAGTGGCCCTGACCGAGACCTGGGCCGGCTCCCA
CTCCATGAGGTATTTCTACACCGCCATGTCCCGGCCCGGCCGCGGGGAGCCCCGCTTCATCGCAGTGGGCTACGTGGACG
ACACCCAGTTCGTGAGGTTCGACAGCGACGCCGCGAGTCCGAGGATGGCGCCCCGGGCGCCATGGATAGAGCAGGAGGGG
CCGGAGTATTGGGACGGGGAGACACGGAACATGAAGGCCTCCGCGCAGACTTACCGAGAGAACCTGCGGATCGCGCTCCG
CTACTACAACCAGAGCGAGGCCGGGTCTCACATCATCCAGGTGATGTATGGCTGCGACGTGGGGCCGGACGGGCGCCTCC
TCCGCGGGCATGACCAGTCCGCCTACGACGGCAAGGATTACATCGCCCTGAACGAGGACCTGAGCTCCTGGACCGCGGCG
GACACGGCGGCTCAGATCACCCAGCGCAAGTGGGAGGCGGCCCGTGTGGCGGAGCAGCTGAGAGCCTACCTGGAGGGCCT
GTGCGTGGAGTGGCTCCGCAGATACCTGGAGAACGGGAAGGAGACGCTGCAGCGCGCGGACCCCCCAAAGACACATGTGA
CCCACCACCCCATCTCTGACCATGAGGCCACCCTGAGGTGCTGGGCCCTGGGCTTCTACCCTGCGGAGATCACACTGACC
TGGCAGCGGGATGGCGAGGACCAAACTCAGGACACCGAGCTTGTGGAGACCAGACCAGCAGGAGATAGAACCTTCCAGAA
GTGGGCAGCTGTGGTGGTGCCTTCTGGAGAAGAGCAGAGATACACATGCCATGTACAGCATGAGGGGCTGCCAAAG0000
TCACCCTGAGATGGGAGCCATCTTCCCAATCCACCGTCCCCATCGTGGGCATTGTTGCTGGCCTGGCTGTCCTAGCAGTT
GTGGTCATCGGAGCTGTGGTCGCTGCTGTGATGTGTAGGAGGAAGAGCTCAGGTGGAAAAGGAGGGAGCTACTCTCAGGC
TGCGTGCAGCGACAGTGCCCAGGGCTCTGATGTGTCTCTCACAGCTTGA
>imgthla|HLA01520|HLA01520 HLA-B*57:01:02, Human MHC Class I sequence (partial)
(SEQ ID NO: 27)
GCTCCCACTCCATGAGGTATTTCTACACCGCCATGTCCCGGCCCGGCCGCGGGGAGCCCCGCTTCATCGCAGTGGGCTAC
GTGGACGACACCCAGTTCGTGAGGTTCGACAGCGACGCCGCGAGTCCGAGGATGGCGCCCCGGGCGCCATGGATAGAGCA
GGAGGGGCCGGAGTATTGGGACGGGGAGACACGGAACATGAAGGCCTCCGCGCAGACTTACCGAGAGAACCTGCGGATCG
CGCTCCGCTACTACAACCAGAGCGAGGCCGGGTCTCACATCATCCAGGTGATGTATGGCTGCGACGTGGGGCCGGACGGG
CGCCTCCTCCGCGGGCATGACCAGTCTGCCTACGACGGCAAGGATTACATCGCCCTGAACGAGGACCTGAGCTCCTGGAC
CGCGGCGGACACGGCGGCTCAGATCACCCAGCGCAAGTGGGAGGCGGCCCGTGTGGCGGAGCAGCTGAGAGCCTACCTGG
AGGGCCTGTGCGTGGAGTGGCTCCGCAGATACCTGGAGAACGGGAAGGAGACGCTGCAGCGCGCGG
>imgthla|HLA02259|HLA02259 HLA-B*57:01:03, Human MHC Class I sequence (partial)
(SEQ ID NO: 28)
GCTCCCACTCCATGAGGTATTTCTACACCGCCATGTCCCGGCCCGGCCGCGGGGAGCCCCGCTTCATCGCAGTGGGCTAC
GTGGACGACACCCAGTTCGTGAGGTTCGACAGCGACGCCGCGAGTCCGAGGATGGCGCCCCGGGCGCCATGGATAGAGCA
GGAGGGGCCGGAGTATTGGGACGGGGAGACACGGAACATGAAGGCCTCCGCGCAGACTTACCGAGAGAACCTGCGGATCG
CGCTCCGCTACTACAACCAGAGCGAGGCCGGGTCTCACATCATCCAGGTGATGTATGGCTGCGACGTGGGGCCGGACGGG
CGCCTCCTCCGCGGGCATGACCAGTCCGCCTACGACGGCAAGGATTACATCGCCCTGAACGAGGACCTGAGCTCCTGGAC
CGCGGCGGACACGGCGGCTCAGATCACCCAGCGCAAGTGGGAGGCGGCCCGTGTGGCGGAGCAGCTGAGAGCCTACCTGG
AGGGCCTGTGTGTGGAGTGGCTCCGCAGATACCTGGAGAACGGGAAGGAGACGCTGCAGCGCGCGG
>imgthla|HLA03969|HLA003969 HLA-B*57:01:04, Human MHC Class I sequence (partial)
(SEQ ID NO: 29)
GCTCCCACTCCATGAGGTATTTCTACACCGCCATGTCCCGGCCCGGCCGCGGGGAGCCCCGCTTCATCGCAGTGGGCTAC
GTGGACGACACCCAGTTCGTGAGGTTCGACAGCGACGCCGCGAGTCCGAGGATGGCGCCCCGGGCACCATGGATAGAGCA
GGAGGGGCCGGAGTATTGGGACGGGGAGACACGGAACATGAAGGCCTCCGCGCAGACTTACCGAGAGAACCTGCGGATCG
CGCTCCGCTACTACAACCAGAGCGAGGCCGGGTCTCACATCATCCAGGTGATGTATGGCTGCGACGTGGGGCCGGACGGG
CGCCTCCTCCGCGGGCATGACCAGTCCGCCTACGACGGCAAGGATTACATCGCCCTGAACGAGGACCTGAGCTCCTGGAC
CGCGGCGGACACGGCGGCTCAGATCACCCAGCGCAAGTGGGAGGCGGCCCGTGTGGCGGAGCAGCTGAGAGCCTACCTGG
AGGGCCTGTGCGTGGAGTGGCTCCGCAGATACCTGGAGAACGGGAAGGAGACGCTGCAGCGCGCGG
>imgthla|HLA04060|HLA04060 HLA-B*57:01:05, Human MHC Class I sequence (partial)
(SEQ ID NO: 30)
GCTCCCACTCCATGAGGTATTTCTACACCGCCATGTCCCGGCCCGGCCGCGGGGAGCCCCGCTTCATCGCAGTGGGCTAC
GTGGACGACACCCAGTTCGTGAGGTTCGACAGCGACGCCGCGAGTCCGAGGATGGCGCCCCGGGCGCCATGGATAGAGCA
GGAGGGGCCGGAGTATTGGGACGGGGAGACACGGAACATGAAGGCCTCCGCGCAGACTTACCGAGAGAACCTGCGGATCG
CGCTCCGCTACTACAACCAGAGCGAGGCCGGGTCTCACATCATCCAGGTGATGTATGGCTGCGACGTGGGGCCGGACGGG
CGCCTCCTCCGCGGGCATGACCAGTCCGCCTACGACGGCAAGGATTACATCGCCCTGAACGAGGACCTGAGTTCCTGGAC
CGCGGCGGACACGGCGGCTCAGATCACCCAGCGCAAGTGGGAGGCGGCCCGTGTGGCGGAGCAGCTGAGAGCCTACCTGG
AGGGCCTGTGCGTGGAGTGGCTCCGCAGATACCTGGAGAACGGGAAGGAGACGCTGCAGCGCGCGG
>imgthla|HLA04456|HLA04456 HLA-B*57:01:06, Human MHC Class I sequence (partial)
(SEQ ID NO: 31)
GCTCCCACTCCATGAGGTATTTCTACACCGCCATGTCCCGGCCCGGCCGCGGGGAGCCCCGCTTCATCGCAGTGGGCTAC
GTGGACGACACCCAGTTCGTGAGGTTCGACAGCGACGCCGCGAGTCCGAGGATGGCGCCCCGGGCGCCATGGATAGAGCA
GGAGGGGCCGGAGTATTGGGACGGGGAGACACGGAACATGAAGGCCTCCGCGCAGACTTACCGAGAGAACCTGCGGATCG
CGCTCCGCTACTACAACCAGAGCGAGGCCGGGTCTCACATCATCCAGGTGATGTATGGCTGCGACGTGGGGCCGGACGGG
CGCCTCCTCCGCGGGCATGACCAGTCCGCCTACGACGGCAAGGATTACATCGCCCTGAACGAGGACCTGAGCTCCTGGAC
CGCGGCGGACACGGCGGCTCAGATCACCCAGCGCAAGTGGGAGGCGGCCCGTGTGGCGGAGCAGCTGAGAGCCTACCTGG
AGGGCCTGTGCGTGGAGTGGCTCCGCAGATACCTGGAAAACGGGAAGGAGACGCTGCAGCGCGCGG
>imgthla|HLA04755|HLA04755 HLA-B*57:01:07, Human MHC Class I sequence
(SEQ ID NO: 32)
GCTCCCACTCCATGAGGTATTTCTACACCGCCATGTCCCGGCCCGGCCGCGGGGAGCCCCGCTTCATCGCAGTGGGCTAC
GTGGACGACACCCAGTTCGTGAGGTTCGACAGCGACGCCGCGAGTCCGAGGATGGCGCCCCGGGCGCCATGGATAGAGCA
GGAGGGGCCGGAGTATTGGGACGGGGAGACACGGAACATGAAGGCCTCCGCGCAGACTTACCGAGAGAACCTGCGGATCG
CGCTCCGCTACTACAACCAGAGCGAGGCCGGGTCTCACATCATCCAGGTGATGTATGGCTGCGACGTGGGGCCGGACGGG
CGCCTCCTCCGCGGGCATGACCAGTCCGCCTACGACGGCAAGGATTACATCGCCCTGAACGAGGACCTGAGCTCCTGGAC
CGCCGCGGACACGGCGGCTCAGATCACCCAGCGCAAGTGGGAGGCGGCCCGTGTGGCGGAGCAGCTGAGAGCCTACCTGG
AGGGCCTGTGCGTGGAGTGGCTCCGCAGATACCTGGAGAACGGGAAGGAGACGCTGCAGCGCGCGGACCCCCCAAAGACA
CATGTGACCCACCACCCCATCTCTGACCATGAGGCCACCCTGAGGTGCTGGGCCCTGGGCTTCTACCCTGCGGAGATCAC
ACTGACCTGGCAGCGGGATGGCGAGGACCAAACTCAGGACACCGAGCTTGTGGAGACCAGACCAGCAGGAGATAGAACCT
TCCAGAAGTGGGCAGCTGTGGTGGTGCCTTCTGGAGAAGAGCAGAGATACACATGCCATGTACAGCATGAGGGGCTGCCA
AAGCCCCTCACCCTGAGATGGG
>imgthla|HLA05320|HLA05320 HLA-B*57:01:08, Human MHC Class I sequence (partial)
(SEQ ID NO: 33)
GCTCCCACTCCATGAGGTATTTCTACACCGCCATGTCCCGGCCCGGCCGCGGGGAGCCCCGCTTCATCGCAGTGGGCTAC
GTGGACGACACCCAGTTCGTGAGGTTCGACAGCGACGCCGCGAGTCCGAGGATGGCGCCCCGGGCGCCATGGATAGAGCA
GGAGGGGCCGGAGTATTGGGACGGGGAGACACGGAACATGAAGGCCTCCGCGCAGACTTACCGAGAGAACCTGCGGATCG
CGCTCCGATACTACAACCAGAGCGAGGCCGGGTCTCACATCATCCAGGTGATGTATGGCTGCGACGTGGGGCCGGACGGG
CGCCTCCTCCGCGGGCATGACCAGTCCGCCTACGACGGCAAGGATTACATCGCCCTGAACGAGGACCTGAGCTCCTGGAC
CGCGGCGGACACGGCGGCTCAGATCACCCAGCGCAAGTGGGAGGCGGCCCGTGTGGCGGAGCAGCTGAGAGCCTACCTGG
AGGGCCTGTGCGTGGAGTGGCTCCGCAGATACCTGGAGAACGGGAAGGAGACGCTGCAGCGCGCGG
>imgthla|HLA05465|HLA05465 HLA-B*57:01:09, Human MHC Class I sequence (partial)
(SEQ ID NO: 34)
GCTCCCACTCCATGAGGTATTTCTACACCGCCATGTCCCGGCCCGGCCGCGGGGAGCCCCGCTTCATCGCAGTGGGCTAC
GTGGACGACACGCAGTTCGTGAGGTTCGACAGCGACGCCGCGAGTCCGAGGATGGCGCCCCGGGCGCCATGGATAGAGCA
GGAGGGGCCGGAGTATTGGGACGGGGAGACACGGAACATGAAGGCCTCCGCGCAGACTTACCGAGAGAACCTGCGGATCG
CGCTCCGCTACTACAACCAGAGCGAGGCCGGGTCTCACATCATCCAGGTGATGTATGGCTGCGACGTGGGGCCGGACGGG
CGCCTCCTCCGCGGGCATGACCAGTCCGCCTACGACGGCAAGGATTACATCGCCCTGAACGAGGACCTGAGCTCCTGGAC
CGCGGCGGACACGGCGGCTCAGATCACCCAGCGCAAGTGGGAGGCGGCCCGTGTGGCGGAGCAGCTGAGAGCCTACCTGG
AGGGCCTGTGCGTGGAGTGGCTCCGCAGATACCTGGAGAACGGGAAGGAGACGCTGCAGCGCGCGG
>imgthla|HLA05563|HLA05563 HLA-B*57:01:10, Human MHC Class I sequence (partial)
(SEQ ID NO: 35)
GCTCCCACTCCATGAGGTATTTCTACACCGCCATGTCCCGGCCCGGCCGCGGGGAGCCCCGCTTCATCGCAGTGGGCTAC
GTGGACGACACCCAGTTCGTGAGGTTCGACAGCGACGCCGCGAGTCCGAGGATGGCGCCCCGGGCGCCATGGATAGAGCA
GGAGGGGCCGGAGTATTGGGACGGGGAGACACGGAACATGAAGGCCTCCGCGCAGACTTACCGAGAGAACCTGCGGATCG
CGCTCCGCTACTACAACCAGAGCGAGGCCGGGTCTCACATCATCCAGGTGATGTATGGCTGCGACGTCGGGCCGGACGGG
CGCCTCCTCCGCGGGCATGACCAGTCCGCCTACGACGGCAAGGATTACATCGCCCTGAACGAGGACCTGAGCTCCTGGAC
CGCGGCGGACACGGCGGCTCAGATCACCCAGCGCAAGTGGGAGGCGGCCCGTGTGGCGGAGCAGCTGAGAGCCTACCTGG
AGGGCCTGTGCGTGGAGTGGCTCCGCAGATACCTGGAGAACGGGAAGGAGACGCTGCAGCGCGCGG
>imgthla|HLA06363|HLA06363 HLA-B*57:01:11, Human MHC Class I sequence
(SEQ ID NO: 36)
GCTCCCACTCCATGAGGTATTTCTACACCGCCATGTCCCGGCCCGGCCGCGGGGAGCCCCGCTTCATCGCAGTGGGCTAC
GTGGACGACACCCAGTTCGTGAGGTTCGACAGCGACGCCGCGAGTCCGAGGATGGCGCCCCGGGCGCCATGGATAGAGCA
GGAGGGGCCGGAGTATTGGGACGGGGAGACACGGAACATGAAGGCCTCCGCGCAGACTTACCGAGAGAACCTGCGGATCG
CGCTCCGCTACTACAACCAGAGCGAGGCCGGGTCTCACATCATCCAGGTGATGTATGGCTGCGACGTGGGGCCGGACGGG
CGCCTCCTCCGCGGGCATGACCAGTCCGCCTACGACGGCAAGGATTACATCGCCCTGAACGAGGACCTGAGCTCCTGGAC
CGCGGCGGACACGGCGGCTCAGATCACCCAGCGCAAGTGGGAGGCGGCCCGTGTGGCGGAGCAGCTGAGAGCCTACCTGG
AGGGCCTGTGCGTGGAGTGGCTCCGCAGATACCTGGAGAACGGGAAGGAGACGCTGCAGCGCGCGGACCCCCCAAAGACA
CATGTGACCCACCACCCTATCTCTGACCATGAGGCCACCCTGAGGTGCTGGGCCCTGGGCTTCTACCCTGCGGAGATCAC
ACTGACCTGGCAGCGGGATGGCGAGGACCAAACTCAGGACACCGAGCTTGTGGAGACCAGACCAGCAGGAGATAGAACCT
TCCAGAAGTGGGCAGCTGTGGTGGTGCCTTCTGGAGAAGAGCAGAGATACACATGCCATGTACAGCATGAGGGGCTGCCA
AAGCCCCTCACCCTGAGATGGG
>EDNA_F
(SEQ ID NO: 37)
CAG ATT TGG ACC TGC GAG CG
>EDNA_R
(SEQ ID NO: 38)
AGG TGA GCG GCT GTC TCC ACA
>EDNA_P
(SEQ ID NO: 39)
TTC TGA CCT GAA GGC TCT GCG CGG
>B51_F
(SEQ ID NO: 40)
GAG TAT TGG GAC CGG AAC ACA
>B51_R
(SEQ ID NO: 41)
AGC CAT ACA TCG TCT GCC AAG T
>B51_P
(SEQ ID NO: 42)
AGA ACC TGC GGA TCG CGC TCC
>B57_F
(SEQ ID NO: 43)
GTC TCA CAT CAT CCA GGT GAT GTA TG
>B57_R
(SEQ ID NO: 44)
GGT GAT CTG AGC CGC CGT
>B57_P
(SEQ ID NO: 45)
CCT CCG CGG GCA TGA CCA GTC

What is claimed is:

1. A method for diagnosing of patients having Behçet's disease or being at risk of a hypersensitivity reaction to abacavir by determining the presence or absence of one or more HLA alleles, the method comprising amplifying and detecting one or more HLA alleles using a real-time polymerase chain reaction (PCR).

2. The method of claim 1 wherein the amplifying is performed using one or more oligonucleotides having binding specificity for HLA-B*51 for diagnosing Behçet's disease or HLA-B*57:01 for identifying a patient as being at risk of a hypersensitivity reaction to abacavir.

3. The method of claim 1 or 2 wherein the one or more oligonucleotides having binding specificity for HLA-B*51 is SEQ ID NO. 40 and/or 41.

4. The method of claim 1 or 2 wherein the one or more oligonucleotides having binding specificity for HLA-B*57:01 is SEQ ID NOS. 43 and/or 44.

5. The method of any one of claims 1-4 wherein the detecting is performed using a reporter oligonucleotide.

6. The method of claim 5 wherein the reporter oligonucleotide has binding specificity for at least a portion of exon 2 and/or exon 3 of a HLA-B*51 allele.

7. The method of claim 5 wherein the reporter oligonucleotide has binding specificity for at least a portion of exon 3 of a HLA-B*57:01 allele.

8. A method for determining the presence or absence of HLA-B*51 allele in a biological sample of an individual, the method comprising:
a) amplifying from the sample a nucleic acid molecule representing at least a portion of exon 2 and/or exon 3 a HLA-B*51 allele; and,
b) detecting amplification of the nucleic acid;
wherein amplification of the nucleic acid indicates that the individual expresses an HLA-B*51 allele.

9. The method of claim 8, wherein step b) comprises hybridizing the amplified nucleic acid fragments to one or more nucleic acid probes specifically hybridizing to target regions within the nucleic acid fragments, the target regions comprising one or more polymorphic nucleotides in exon 2 and/or 3 of HLA-B*51.

10. The method of claim 8 or 9 wherein the amplification is performed using real-time polymerase chain reaction (PCR) with forward and reverse primers having specificity for one or more HLA-B*51 alleles.

11. The method of any one of claims 8-10 wherein the amplified nucleic acid corresponds to an HLA-B*51 subtype selected from the group consisting of HLA-B*51:01 (SEQ ID NO: 1), HLA-B*51:02 (SEQ ID NO: 2), HLA-B*51:03 (SEQ ID NO: 3), HLA-B*51:05 (SEQ ID NO: 4), HLA-B*51:07 (SEQ ID NO: 5), HLA-B*51:08 (SEQ ID NO: 6), HLA-B*51:09 (SEQ ID NO: 7), HLA-B*51:10 (SEQ ID NO: 8), HLA-B*51:11N (SEQ ID NO: 9), HLA-B*51:12 (SEQ ID NO: 10), HLA-B*51:13 (SEQ ID NO: 11) HLA-B*51:14 (SEQ ID NO: 12), HLA-B*51:15 (SEQ ID NO: 13), HLA-B*51:21 (SEQ ID NO: 14), HLA-B*51:22 (SEQ ID NO: 15), HLA-B*51:26 (SEQ ID NO: 16), HLA-B*51:27N (SEQ ID NO: 17), HLA-B*51:29 (SEQ ID NO: 18), HLA-B*51:31 (SEQ ID NO: 19), HLA-B*51:32 (SEQ ID NO: 20), HLA-B*51:33 (SEQ ID NO: 21), HLA-B*51:36 (SEQ ID NO: 22), HLA-B*51:37 (SEQ ID NO: 23), HLA-B*51:63 (SEQ ID NO: 24) and/or HLA-B*51:65 (SEQ ID NO: 25), and/or a fragment and/or derivative thereof.

12. The method of claim 10 wherein the forward primer is SEQ ID NO. 40, the reverse primer is SEQ ID NO:41, and/or the forward primer is SEQ ID NO. 40 and the reverse primer is SEQ ID NO:41.

13. A method for determining the presence or absence of HLA-B*57:01 allele in a biological sample of an individual, the method comprising:
a) amplifying from the sample a nucleic acid representing at least a portion of exon 3 of a HLA-B*57:01 allele; and,
b) detecting amplification of the nucleic acid;
wherein amplification of the nucleic acid indicates that the individual expresses a HLA-B*57:01 allele.

14. The method of claim 13, wherein step b) comprises hybridizing the amplified nucleic acid fragments to one or more nucleic acid probes specifically hybridizing to target regions within the nucleic acid fragments, the target regions comprising one or more polymorphic nucleotides in exon 3 of one or more HLA-B*57:01 alleles.

15. The method of claim 13 or 14 wherein the amplification is performed using real-time polymerase chain reaction (PCR) with forward and reverse primers having specificity for one or more HLA-B*57:01 alleles.

16. The method of any one of claims 13-15 wherein the amplified nucleic acid corresponds to an HLA-B*57 subtype selected from the group consisting of HLA-B*57:01:01 (SEQ ID NO: 26), HLA-B*57:01:02 (SEQ ID NO: 27), HLA-B*57:01:03 (SEQ ID NO: 28), HLA-B*57:01:04 (SEQ ID NO: 29), HLA-B*57:01:05 (SEQ ID NO: 30), HLA-B*57:01:06 (SEQ ID NO: 31), HLA-B*57:01:07 (SEQ ID NO: 32), HLA-B*57:01:08 (SEQ ID NO: 33), HLA-B*57:01:09 (SEQ ID NO: 34), HLA-B*57:01:10 (SEQ ID NO: 35), and HLA-B*57:01:11 (SEQ ID NO: 36), and/or a fragment and/or derivative thereof.

17. The method of claim 15 wherein the forward primer is SEQ ID NO.:43, the reverse primer is SEQ ID NO.:44, and/or the forward primer is SEQ ID NO.:43 and the reverse primer is SEQ ID NO.:44.

18. The method of claim 9 or 14 where the nucleic acid probes are SEQ ID NO.:42 or SEQ ID NO.:45, respectively.

19. The method of claim 9, 14 or 18 wherein the one or more nucleic acid probes are labeled with a detectable label.

20. The method of any one of claims 1-18 wherein the real time PCR is single or multiplex assay.

21. The method of any one of claim 19 wherein the real time PCR includes the amplification of a control nucleic acid sequence.

22. A kit for typing HLA-B*51 alleles in a biological sample of an individual, the kit comprising oligonucleotides having the sequence of SEQ ID NOS.: 40 and/or 41.

23. A kit for typing HLA-B*57 alleles in a biological sample of an individual, the kit comprising oligonucleotides SEQ ID NOS.: 43 and/or 44.

24. The kit of claim 21 or 22 further comprising a control sample and/or control oligonucleotides.

25. The kit of any one of claims 21-23 further comprising one or more oligonucleotide probes for detecting an amplified nucleic acid molecule corresponding to the allele.

26. The kit of claim 25 where the one or more oligonucleotide acid probes comprises SEQ ID NO.:42 or SEQ ID NO.:45.

27. The kit of claim 25 or 26 wherein the one or more oligonucleotide probes comprises a detectable label.

28. The kit of any one of claims 21-27 further comprising instructions for use.
